(19) United States
(12) Patent Application Publication
(10) Pub. No.: US 2023/0390562 A1
(43) Pub. Date: Dec. 7, 2023

(54) A CATHETER AND METHOD FOR DETECTING DYSSYNERGY RESULTING FROM DYSSYNCHRONY

(71) Applicant: Pacertool AS, Oslo (NO)

(72) Inventor: Hans Henrik ODLAND, Oslo (NO)

(21) Appl. No.: 18/031,846

(22) PCT Filed: Oct. 13, 2021
(86) PCT No.: PCT/EP2021/078365

§ 371 Date: Apr. 13, 2023

(30) Foreign Application Priority Data
Oct. 13, 2020 (GB) 2016234.3

(51) Int. Cl. A61N 1/365; A61N 1/368; A61N 1/37; A61M 25/00; A61B 5/00

(52) U.S. Cl. CPC A61N 1/36514; A61N 1/36842; A61N 1/3704; A61N 1/3686; A61M 25/0053; A61B 5/6846; A61B 5/72; A61M 2025/0001

(57) ABSTRACT

There is provided a catheter for assessing cardiac function, the catheter comprising an elongate shaft extending from a proximal end to a distal end, where the shaft comprises a lumen for a guidewire and/or a saline flush. The catheter further comprises at least one electrode disposed on the shaft for sensing electrical signals in a bipolar or unipolar fashion and applying pacing to a patient's heart, at least one sensor disposed on the shaft for detecting an event relating to the rapid increase in the rate of pressure increase within the left ventricle of a patient; and communication means configured to transmit data received from the electrode(s) and the sensor(s).

### TECHNICAL FIELD

The present invention is concerned with a catheter that may be utilised in a system and a method for detecting dyssynergy resulting from dyssynchrony, a system and method for determining optimal electrode number and positions for cardiac resynchronisation therapy and/or a method and system for measuring time to fusion as a means of determining degree of parallel activation of the heart. Thus, the invention may be used in relation to patient's suffering dyssynchronous heart failure, and more specifically can apply to the identification of patients who are likely to respond to resynchronization therapy, as well as optionally determining optimal locations for placement of electrodes to stimulate the heart. The invention may also be used for patients who have suffered dyssynchronous heart failure.

### BACKGROUND OF THE INVENTION

Cardiac resynchronization therapy (CRT) is consistently provided according to recognized medical standards and guidelines provided by international medical societies in order to treat patients suffering from various conditions such as a widened QRS complex, (left or right) bundle branch block and heart failure. There are some minor differences between the medical guidelines regarding the specific conditions that should occur before CRT is utilized, such as how wide the QRS complex is, what type of bundle branch block is being suffered and the degree of heart failure.

CRT is associated with a reduction in mortality and morbidity; however, not all patients benefit from such therapy. In fact, some patients may experience deterioration after treatment, some experience devastating complications, and some experience both.

In this regard, it would be beneficial to provide a unifying strategy that reduces the number of non-responders to CRT and optimize the treatment of potential responders, and therefore increases the effectiveness of therapy.

### SUMMARY OF THE INVENTION

Viewed from a first aspect, the present invention provides a catheter for assessing cardiac function, the catheter comprising
an elongate shaft extending from a proximal end to a distal end, the shaft comprising:
   a lumen for a guidewire and/or a saline flush;
at least one electrode disposed on the shaft for sensing electrical signals in a bipolar or unipolar fashion and applying pacing to a patient's heart;
at least one sensor disposed on the shaft for detecting an event relating to the rapid increase in the rate of pressure increase within the left ventricle of a patient; and
communication means configured to transmit data received from the electrode(s) and the sensor(s).

As discussed below, such a catheter may fund particular use when determining function of the heart, and particularly when providing measures indicating whether dyssynergy resulting from dyssynchrony is present within a patient. When the catheter is suitably positioned in the left heart chamber with electrodes opposing each other at the septum and contralateral wall and the sensor within the chamber, with each heartbeat a voltage gradient is registered between each electrode and a reference electrode. Such a voltage gradient represents electric activation of the heart at the site of the electrode. The time course of activation of the different electrodes determines the degree of dyssynchrony. Further, and following on from the above, the sensor(s) register events related to the onset of synergy, i.e. events that relate to the rapid increase in rate of pressure rise within the left ventricle, which reflects the point where all segments of the heart begin to actively or passively stiffen. The time to this event is compared with electrical activation and the degree of dyssynchrony, and the presence of dyssynergy resulting from dyssynchrony is registered. Whilst herein the rapid increase in pressure of the left ventricle is referred to, the skilled person would understand that such an event could manifest in a more general in pressure within the heart of a patient. In this way, the catheter may not necessarily be placed within the left ventricle of the patient.

The heart can then be stimulated from one or more electrode. With each heartbeat a voltage gradient is registered between each electrode and a reference electrode, which as described above can represent the electric activation of the heart. The one or more sensor again registers events related to the onset of synergy. The new set of time events may then be compared to the first set of events and the presence or absence of resynchronization is registered.

Advantageously, with such a system, it may be possible to quickly and efficiently determine such measures for various positions of electrodes. In this way, not only may it be determined if a patient is indeed a potential responder for cardiac resynchronisation therapy, but also the ideal number and positions of electrodes may be quickly determined.

The at least one sensor comprises a pressure sensor, a piezoelectric sensor, a fiberoptic sensor, and/or an accelerometer. Such sensors can find particular use in detecting events relating to the rapid increase in the rate of pressure increase in the left ventricle, as further discussed below.

The stiffness of the elongate shaft may vary along its length between the proximal end and the distal end. In this way the elongate shaft may have a structure that is ideal for quick and easy positioning within the patient's heart. Optionally, the elongate shaft is provided with a stiff proximal end, a middle part which is of an intermediate stiffness, and a flexible tip at the distal end. Again, such a structure provides for a catheter that may be easily manoeuvred within the heart.

The at least one electrode may comprise a plurality of electrodes disposed along the shaft such that, in use, at least two electrodes may be positioned opposing each other in the heart of the patient. Optionally, the at least one electrode is configured to be placed within the septum of the patient, and at least one electrode is configured to be placed in the contralateral wall of the patient.

In a second aspect, there is provided a system comprising
the catheter as described above;
a signal amplifier;
a stimulator; and
a data processing module;
wherein the catheter is configured to be in signal communication with the stimulator, the amplifier and data processing module such that the electrode(s) and sensor(s) may provide sensed data to the data processing module for further processing, and the electrode(s) may provide pacing to the patient's heart.

Such a system may be utilised to quickly and easily determine how moving the catheter about the heart, and therefore moving the attached electrodes effects the functioning of the heart, and particularly whether pacing makes any marked difference in reducing dyssynchrony and/or dyssynergy.

The data processing module is configured to determine a characteristic response relating to the onset of myocardial synergy from the event relating to the rapid increase in the rate of pressure increase within the left ventricle of a patient.

The sensor(s) may be any kind of appropriate sensor, or a combination of appropriate sensors, such as an acceleration, rotation, vibration and/or a pressure sensor. The sensor(s) may be configured to provide data regarding the pressure within the heart to the data processing module, and wherein the data processing module is configured to filter the pressure data to identify the characteristic response relating to the onset of myocardial synergy. The characteristic response may comprise the beginning of a pressure rise above the pressure floor in a pressure signal filtered above the first harmonic of the pressure signal. The characteristic response may comprise the presence of high frequency components (above 40 Hz) of the pressure signal. The characteristic response may comprises a band-pass filtered pressure trace crossing zero. By filtering the pressure trace it is possible to remove associated noise and more accurately and reliably determine a point that relates to the onset of myocardial synergy.

Additionally or alternatively, the sensor(s) may be configured to provide acceleration data from within the heart to the data processing module, and the data processing module may be configured to filter the acceleration data to identify a characteristic response relating to the onset of myocardial synergy. For example, the data processing module may be configured to calculate a continuous wavelet transform of the acceleration data to identify a characteristic response relating to the onset of myocardial synergy. The data processing module may be configured to calculate the center frequency of the continuous wavelet transform, wherein the characteristic response is the peak of the center frequency. The data processing module is configured to average the center frequency over a number of heart cycles. By filtering the acceleration trace it is possible to remove associated noise and more accurately and reliably determine a point that relates to the onset of myocardial synergy.

As would be appreciated, in addition or as an alternative to the above, there are provided several further methods herein that enable a characteristic response relating to the onset of myocardial synergy to be determined. The data processing module may be configured to perform one or more of such methods.

For example, the increase of pressure within the heart (for example, pressure within the left ventricle) over time for two different stimuli may be compared. For example, a pressure curve that results from the pacing of the right ventricle and a pressure curve that results from biventricular pacing may be compared. The pressure rises resulting from the two stimuli may be fitted together relative to their stimulation timing, and the pressure level adjusted to fit the diastolic portion of the curves prior to ventricular pacing. The point at which the pressure curve resulting from the stimuli begin to deviate from one another may then be detected, which indicates the time of the onset of synergy of the stimuli that results in the earliest pressure rise.

The portion of the pressure rise curve that follows the time of the onset of synergy on the pressure curve resulting of the stimulus that results in an earlier pressure rise may then be shifted so as to fit on the portion of the pressure rise curve of the stimulus that results in a comparatively delayed pressure rise. The point on the pressure rise curve of the stimulus that results in a comparatively delayed pressure rise at which the curve following the onset of synergy of the stimulus that results in the earlier pressure rise is the point of onset of synergy in the delayed pressure rise curve. The delay may then be calculated between the two determined points of onset of synergy. From such a calculation, a recommendation may be made to which pacing regime should be following in an implanted pacemaker.

The above process may be automated and for the data resulting from any number of pacing regimes/stimuli, whether by a simple matching of the curves (for example, by the fitting of a template to the pressure trajectory with a least squares method) or by a comparison of the mathematical formulae that represent the curves. In this way, an explicit plotting of the pressure curve and a visual matching of the curve may not be necessary, but rather the raw data may be analysed so as to allow for similar conclusions to be reached.

In this way, there can be an automatic detection in the data of the exponential pressure rise, up to the peak dP/dt which results from the onset of synergy. There may be an automatic calculation of the exponential formula that fits the pressure curve, and the time when the exponential formula fits one of a number of curves can be determined.

There may be a template match, and there may be calculated a time offset between the exponential formula and the template matches, or equally a cross-correlation between other measures.

The above method may equally be performed using filtered pressure measurements.

Additionally or alternatively, an advancement of the onset of synergy may be detected by an advancement of the zero-crossing of the band-pass filtered (e.g. 4-40 Hz) pressure curve (Tp) with stimulation from a certain pacing regime compared to another kind of pacing. Such data may be used to indicate the presence of synergy with a certain pacing regime, and therefore that it may be desirable to undergo CRT with that pacing regime.

The method may include calculating a baseline interval (B) by determining a time period between intrinsic atrial activation (Ta) and the associated zero crossing of the resulting pressure curve (Tp). A corresponding time period (Tp1) may be calculated following pacing from a first electrode at a set pacing interval (PI1) after Ta, and the pacing interval reduced until the Ta to Tp interval is less than B. A corresponding time period (Tp2) may be calculated following pacing from a second electrode at a set pacing interval (PI2) after Ta, and the pacing interval reduced until the Ta to Tp interval is less than B. A corresponding time period (Tp3) may be calculated following pacing from the first and second electrodes at a set pacing interval (P13) after Ta, where P13 is the same time interval of the lower of PI1 and PI2. By determining which pacing has the shortest corresponding time period Tp, the pacing regime that leads to the highest degree of synergy may be identified.

The data processing module may be configured to identify reversible cardiac dyssynchrony by identifying a shortening of a delay to onset of myocardial synergy as a result of pacing. Specifically, the data processing module may be configured to identify reversible cardiac dyssynchrony of a patient using the at least one sensor to measure the time of the event relating to the rapid increase in the rate of pressure increase within the left ventricle of a patient by identifying the characteristic response in the data received from the one or more sensors, the event relating to the rapid increase in the rate of pressure increase within the left ventricle being identifiable in each contraction of the heart.

The data processing module may be configured to measure the time of the event relating to the rapid increase in the rate of pressure increase within the left ventricle by;
processing signals from the at least one sensor to determine a first time delay between the measured time of the identified characteristic response relating to the rapid increase in the rate of pressure increase within the left ventricle and a first reference time;
   comparing the first time delay between the measured time of the identified characteristic response relating to the rapid increase in the rate of pressure increase within the left ventricle and the first reference time with the duration of electrical activation of the heart;
if the first time delay is longer than a set fraction of electrical activation of the heart, then identifying the presence of cardiac dyssynchrony in the patient;
following the application of pacing by the at least one electrode and/or other electrodes to the heart of the patient;
calculating a second time delay between the identified characteristic response relating to the rapid increase in the rate of pressure increase within the left ventricle following pacing and a second reference time following pacing by:
   using the at least one sensor to measure the timing of the identified characteristic response relating to the rapid increase in the rate of pressure increase within the left ventricle following pacing; and
   processing signals from the at least one sensor to determine the second time delay between the determined time of the identified characteristic response relating to rapid increase in the rate of pressure increase within the left ventricle and the second reference time following pacing;
comparing the first time delay and the second time delay; and
if the second time delay is shorter than the first time delay, identifying a shortening of a delay to onset of myocardial synergy, OoS, indicating that the time period until the point where all segments of the heart begin to actively or passively stiffen has shortened, thereby identifying the presence of reversible cardiac dyssynchrony in the patient.

Further, the data processing module may be configured to determine the degree of parallel activation of a heart undergoing pacing. Specifically the data processing module may be configured to determine the degree of parallel activation of a heart undergoing pacing via a method comprising:
calculating a vectorcadiogram, VCG, or electrocardiogram, ECG, waveforms from right ventricular pacing, RVp, and left ventricular pacing, LVp;
generating a synthetic biventricular pacing, BIVP, waveform pacing by summing the VCG of the RVp and the LVp, or by summing the ECG of the RVp and the LVp;
calculating a corresponding ECG or VCG waveform from real BIVP;
comparing the synthetic BIVP waveform and the real BIVP waveform;
calculating time to fusion by determining the point in time in which the activation from RVp and LVp meets and the synthetic and the real BIVP curves start to deviate;
wherein
a delay in time to fusion indicates that a larger amount of tissue is activated before wave fronts for electrical activation meet, thereby indicating a higher degree of parallel activation.

Further, the data processing module is configured to determine the optimal electrode number and position for cardiac resynchronization therapy on the heart of the patient based on node(s) of a 3D mesh 3D mesh of at least part of the heart with a calculated degree of parallel activation of the myocardium above a predetermined threshold. Specifically, the system may be configured to perform a method determining optimal electrode number and positions for cardiac resynchronization therapy on a heart of a patient, via a method comprising;
generating the 3D mesh of at least part of the heart from a 3D model of at least part of the heart of the patient, or using a generic 3D model of the heart to obtain a 3D mesh of at least a part of the heart, the 3D mesh of at least a part of the heart comprising a plurality of nodes;
aligning the 3D mesh of at least part of a heart to images of the heart of the patient;
placing additional nodes onto the 3d mesh corresponding to a location of at least two electrodes on the patient;
calculating a propagation velocity of the electrical activation between the nodes of the 3D mesh corresponding to the location of the at least two electrodes;
extrapolating the propagation velocity to all of the nodes of the 3D mesh;
calculating the degree of parallel activation of the myocardium for each node of the 3D mesh; and
determining the optimal electrode number and position on the heart of the patient based on the node(s) of the 3D mesh with a calculated degree of parallel activation of the myocardium above a predetermined threshold.

The catheter may be configured to be provided into a patient's heart through arterial access, venous access, subclavian access, radial access and/or femoral access such that the electrode(s) and sensor(s), in use, may be provided within the heart of the patient.

### BRIEF DESCRIPTION OF THE DRAWINGS

Certain preferred embodiments will now be described by way of example only and with reference to the accompanying drawings, in which:

FIG. 1a shows a representation of a normal heart;

FIG. 1b shows a heart undergoing CRT and hence being implanted with atrial and biventricular electrodes;

FIG. 2 illustrates a 3D surface geometry model of the heart with representations of locations of the electrodes of FIG. 1b;

FIG. 3 is an example system for measuring bioimpedance on the heart;

FIG. 4a shows measurements of any representation of onset of synergy along with impedance and/or acceleration;

FIG. 4b shows an echocardiographic representation of time to onset of synergy;

FIG. 5a illustrates how a pressure catheter located within the left ventricle can be utilized to measure ventricular pressure and the derivative of the pressure waveform;

FIG. 5b shows the placement of sonomicrometric crystals in the heart for subsequent measurements of myocardial segmental lengths and stiffness;

FIG. 5c shows such a determination of onset of myocardial synergy and how this relates to measuring a peak in the second-order derivative of left ventricular pressure from the measurement arrangement of FIG. 5b;

FIG. 5d illustrates the change in time to peak dP/dt with a change in position of pacing causing less dyssynchrony (position 2);

FIG. 6 shows an illustration of physiological conditions experienced during heart contraction;

FIG. 7a shows various signals that can be derived from filtering measured traces;

FIG. 7b shows various other traces from filtered waveforms;

FIGS. 8a, 8b and 8c show various examples of how traces may be utilised to determine the onset of synergy, or a signal indicative thereof;

FIG. 9 shows a method for generating a 3D model of the heart including a 3D mesh of the ventricle;

FIG. 10 illustrates the use of x-ray in relation to alignment of the 3D model with the patient's heart;

FIG. 11 shows x-ray images taken for use in the alignment of the 3D model;

FIG. 12 shows reconstruction of the coronary sinus vein in 3D;

FIG. 13a illustrates a heart model converted to a geometric model;

FIG. 13b illustrates another geometric heart model in 3D;

FIG. 14 is a visualization of time propagation of electrical activation;

FIG. 15 shows the use of an object of known size to calibrate the heart model for distance between vertices;

FIG. 16 illustrates pacing of the right ventricle in order to extrapolate measurements of recruited area of the heart;

FIG. 17 shows a similar process to FIG. 16 but using separation time based on natural pacing of the heart;

FIG. 18a shows a calculation of a compound measure, with FIG. 18b showing the addition of geodesic distance and highlighting of areas for potential electrode placement;

FIG. 19 shows an example of calculation of geodesic velocity;

FIG. 20 is a heart model including a representation of propagation of electrical activation from the nodes;

FIG. 21 shows echocardiographic parameters associated with the heart model;

FIG. 22 visualizes tissue characteristics with reference to scar tissue;

FIGS. 23 and 24 show recruitment curves representing the recruited area in the heart model;

FIG. 25a shows a vectorcardiogram (VCG) created for an electrode performing right ventricular pacing (RVp); and

FIG. 25b illustrates a comparison of synthetic VCG LVP+RVp and the real VCG BIVp.

FIG. 26 shows an example catheter FIG. 27 shows a detailed illustration of an example guidewire for use with the catheter of FIG. 26.

FIG. 28 shows how a guidewire is used to manoeuvre the catheter.

FIG. 29 shows various access routes to bring the catheter into the heart.

FIG. 30 shows a cross section of the catheter.

FIG. 31 shows a more detailed view of the structure of the catheter.

FIG. 32 shows a block diagram of system comprising the catheter.

FIG. 33 shows various traces that can be extracted from accelerometer data from an accelerometer sensor positioned within the heart.

FIG. 34 shows in more detail selected traces of FIG. 33.

FIG. 35 shows an example analysis that may be performed to acceleration data, so as to calculate a time to onset of synergy.

FIG. 36 shows a graph of example derivatives of Pₜᵣᵤₑ and P_{reading} to show the sensor calibration effect.

FIG. 37 shows an exemplary catheter, along with some example dimensions over which it may extend.

FIG. 38 shows a comparison of two pressure curves resulting from different kinds of pacing.

FIG. 39a shows various traces in which the advancement of the zero crossing of the pressure curve can be detected.

FIG. 39b shows a more detailed view of the traces of FIG. 39a.

FIG. 40 shows the comparative shortening of onset of synergy and time to peak dP/dt with various kinds of pacing.

FIG. 41 shows a visual representation of an advancement of Td with various kinds of pacing.

### DETAILED DESCRIPTION

Assessment of Cardiac Dyssynchrony

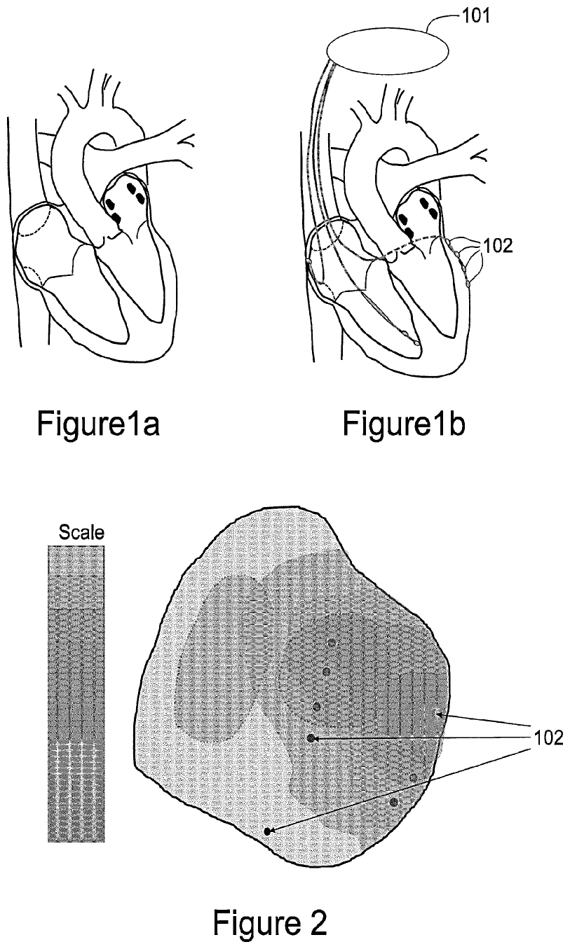

A representation of a normal heart may be seen in FIG. 1a. Typically, a heart undergoing CRT may be implanted with atrial and biventricular electrodes 102 as in FIG. 1b, which are connected to a programmable pacemaker 101.

The locations of said electrodes 102 may be represented on a 3D surface geometry model of the heart, thereby showing a heart model display with colour maps representing measurement zones relative to the electrodes as seen in FIG. 2. A contour map may then be projected onto the surface of the heart model in order to visualize lines of constant magnitude of a measured value at each area of the heart, and the location of the electrodes within the color zones. Each color represents a measurement, and different degrees of colors represent different degrees of that measure as seen in the scale. For example, measurements pertaining to the intracardiac impedance measured between a pair of electrodes may be visualized on such a model in this way.

Firstly, the system may comprise a bioimpedance measurement system is provided to connect to pacing wires that are situated within any chambers and/or vessels of the heart and surface electrodes for current injection. Measurements of complex impedance, phase and amplitude will allow characterization of the time of onset of myocardial synergy.

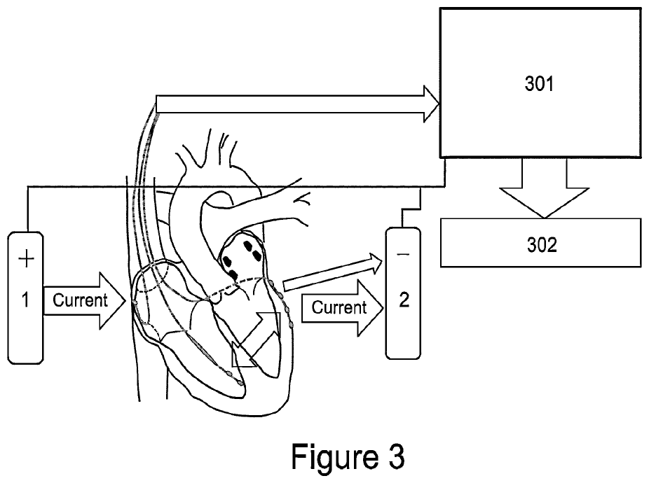

An example system for measuring bioimpedance may be seen in FIG. 3. Therein is shown a measurement setup for impedance (dielectric) measurements on the heart, with implanted CRT electrodes as shown in FIG. 1b. Current may be injected through surface skin electrodes 1 and 2, and impedance may be measured between the electrodes, or between electrodes and patches. Multiple electrodes can be included in measurements of complex impedance. Impedance may then be processed in a processing unit 301, and converted into digital signals that can further be transferred to any digital signal processing unit 302 for display of complex impedance waveforms. The calculated impedance waveforms may further be utilized for calculation of onset of synergy or be compared to known waveforms for similarity or deviation therefrom. Multiple frequencies of injected current may be adjusted to optimize the amplitude phase relationship and directional change for optimization of the impedance phase trajectory interaction.

The electrodes may be placed on the surface of the body, for example perpendicular to the axis of the heart (from center of mitral valve orifice to the LV apex) for current injection. Current injection may also be performed from electrodes located within the heart.

The system may further include one or more sensors to provide measures of onset of synergy as described above. For example, an accelerometer or a piezo-resistive sensor or a fibreoptic sensor may also be provided either on the body surface, or embodied within a catheter in the heart (such as an ablation catheter for detection of the His potential) to detect the heart sounds, aortic valve opening or closure. An ultrasound sensor may be used to provide similar measurements. A pressure transducer may be positioned on a catheter within the right or left ventricle, so as to detect peak pressure rise in the time domain, and/or to detect trajectory advancement. The transducer may also measure any delay compared to any trajectory in either the time derivative of the pressure curve trajectory or in the pressure curve trajectory itself. Additionally, and/or alternatively, surface electrodes for producing an ECG may also be provided.

The data provided by the sensors may then be processed and used to calculate a degree of offset between the onset of pacing and the onset of myocardial synergy as a measure of cardiac dyssynchrony.

For example, a circuit implemented in hardware and/or software is used to receive signals from one or more of the above described sensors and/or measurements, corresponding to the time when the cardiac activation and contraction leads to ejection.

The circuit may then additionally receive the ECG signal of the heart, which corresponds to time point when the heart starts depolarizing, as well as when it is fully depolarized. The ECG can be used as a time reference, and the resulting signals can be related to the onset/offset of intrinsic activation of the heart, and/or onset of pacing as seen in the surface ECG. Such information may be utilized as a reference to provide a time interval relative to onset of pacing and/or onset/offset of the ECG.

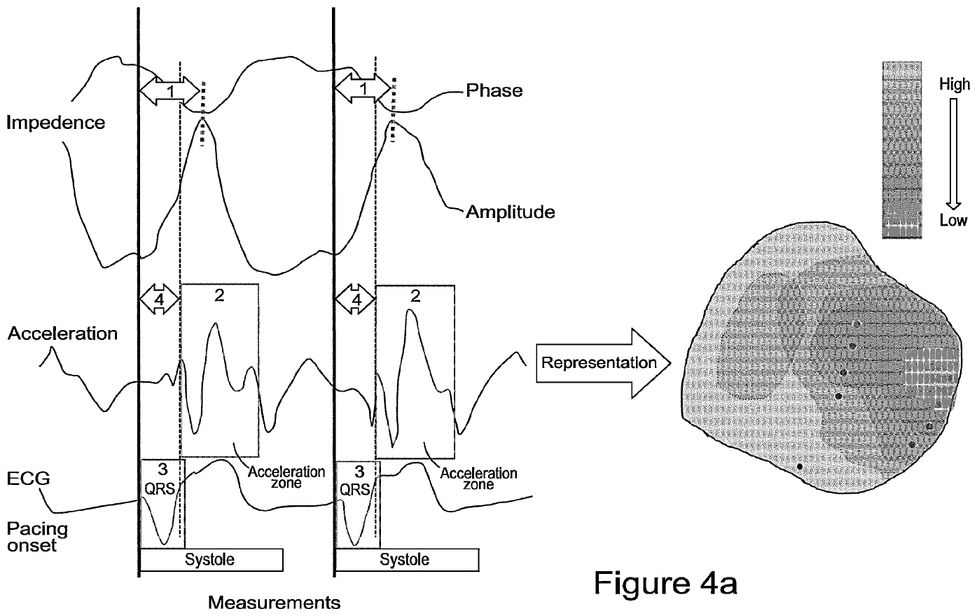

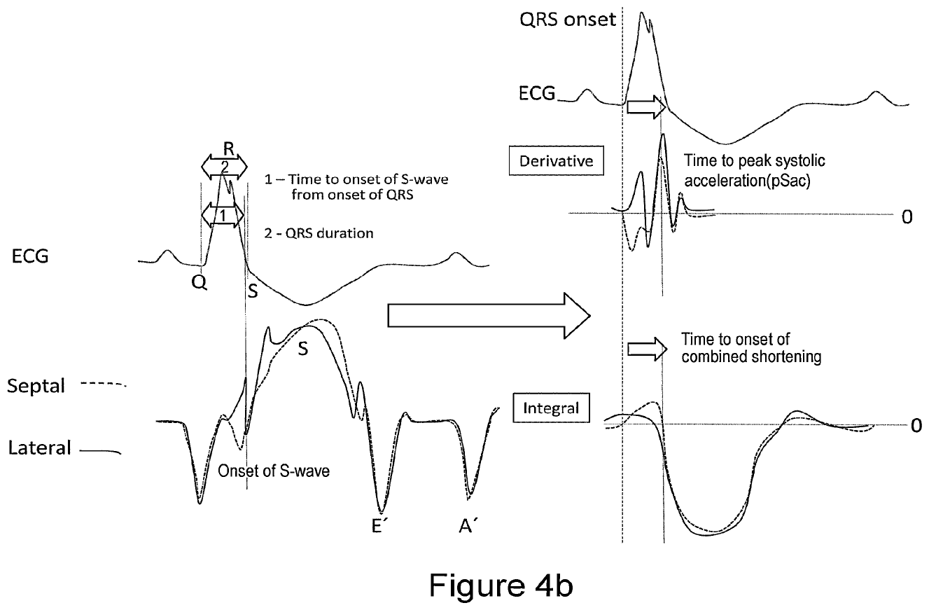

Such a utilization of measurements as a way of measuring the delay to onset of myocardial synergy may be seen in FIG. 4a. FIG. 4a shows measurements of any representation of onset of synergy, measured with impedance and/or acceleration or piezo-resistive sensor signals.

The measured impedance is represented with complex impedance (phase), corresponding to the contraction of the heart muscle, and the amplitude, corresponding to the blood volume within the heart. In this way, the amplitude of the impedance signal may be used as a surrogate for volume changes within the left heart chamber, as changes in the amplitude signal is paralleled by changes in ventricular blood volume. The phase of the impedance is used as a surrogate of muscle contraction, as changes are paralleled by changes in muscle volume and intracardiac blood volume.

The time from a reference point until the impedance curves meet and deviate (1) may be measured as a representation of onset of synergy. Such a point occurs at the point where the muscle shortens and blood is ejected from the heart. Acceleration from any acceleration sensor within (or connected to the surface of) the body of the patient can be used to determine onset of acceleration after a given reference point (4). Any part of the stable acceleration signal that reproduces itself from beat to beat and stimulation site may be used as a representation of onset of synergy. For example, the part of the acceleration signal used to determine the onset of synergy may correspond to any heart sound, aortic valve opening or closure.

Further, the ECG signal can be used as the reference point, from any of onset, offset or full duration of the QRS signal (3), and equally the acceleration signal can be used as a reference (2) from onset, offset or full duration (2). As described above, any such measurements can further be visualized on a surface of a heart geometry using color coded zones and a scale, relative to electrodes.

As would be appreciated, other measurements may be utilized to relate to the onset of synergy, such as measurements of the myocardial acceleration or when using a phonocardiogram or from seismocardiography. For example, echocardiography, sonography and cardiac ultrasound within or from outside the body to may be utilized to measure myocardial wall velocity, strain or any other measure that repeats in each cycle to measure onset of synergy. Specifically, at least one of onset of S-wave velocity, onset of S-wave strain rate, onset of global ejection, aortic valve opening, onset of aortic flow may be measured.

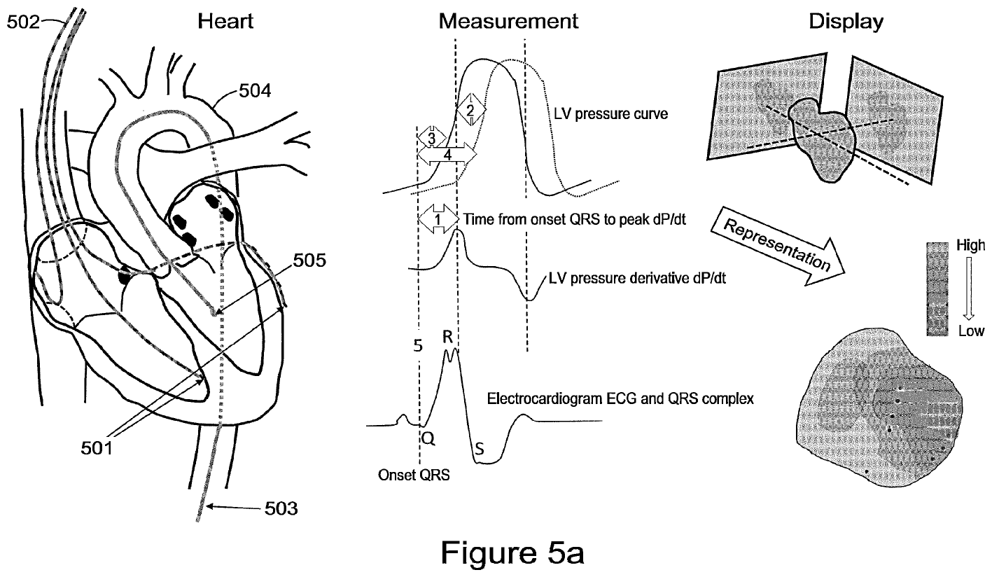

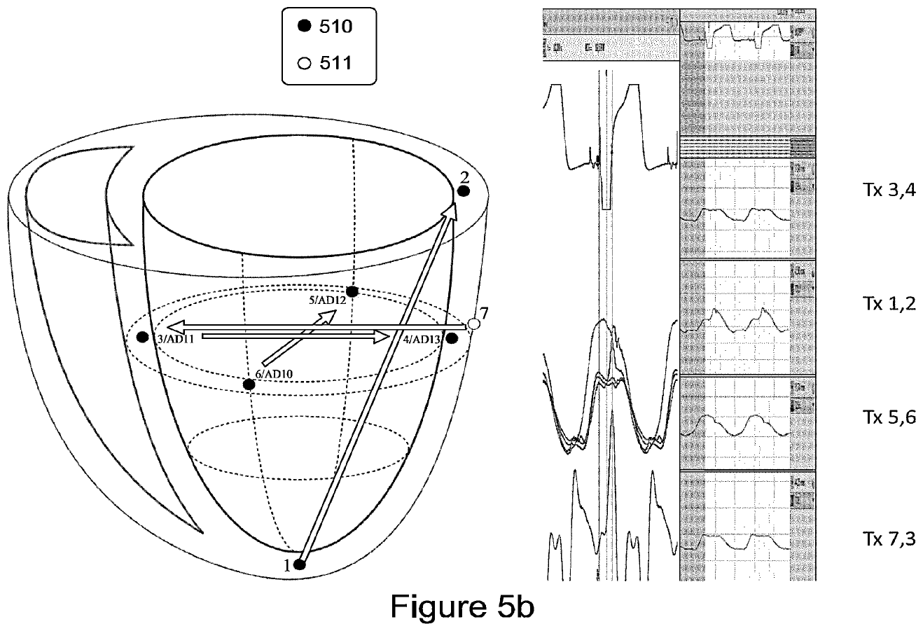

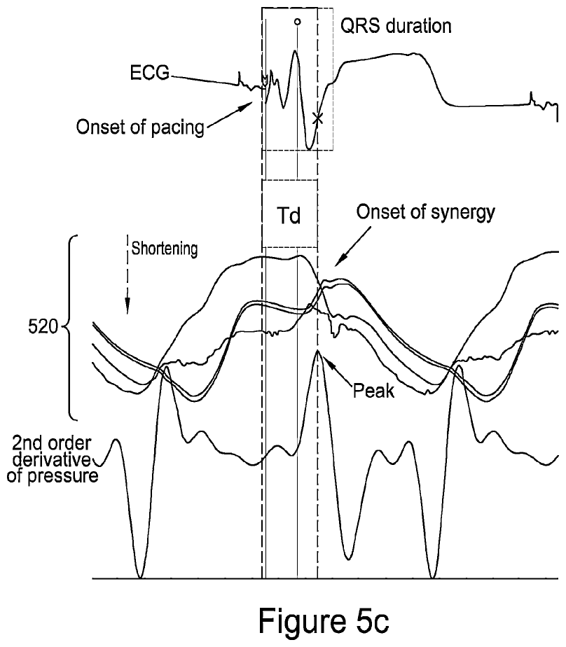

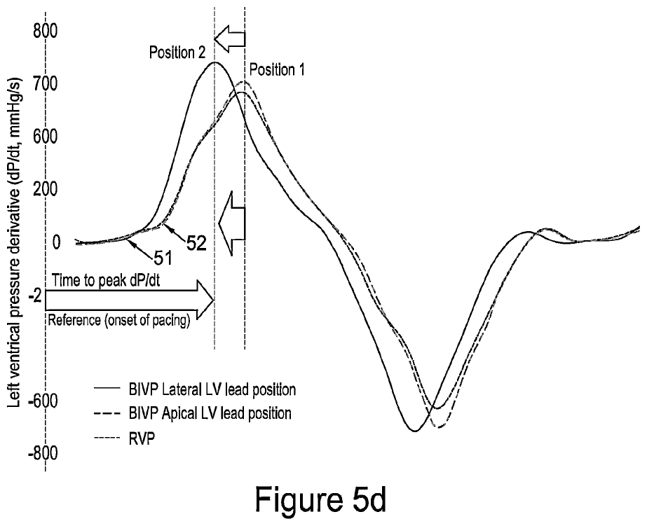

FIG. 4b shows tissue Doppler trajectories processed in an echocardiography device to show tissue velocities, thereby showing an echocardiographic representation of time to onset of synergy, in measures such as the time to onset of the S-wave, pSac and shortening. The echocardiograph may be a representation of septal and lateral tissue velocity, acceleration and displacement. The velocity trajectories have letters assigned to them according to which part of the cardiac cycle (Wiggers diagram) they represent isovolumic contraction (IVC), the systolic velocity (S) and isovolumic relaxation (IVR). Through derivation velocity is converted into acceleration and with integration velocity is converted to displacement. Onset of S-wave and peak systolic acceleration reflects onset of synergy and can be used for determining the time from a reference to onset of synergy as described above. Any event that follows can be used for the same purpose. When strain or strain rate is calculated measurements can be performed in a similar fashion. In another example, using the system described above, myocardial dyssynchrony may be measured in the form of the time from pacing spike and/or QRS onset/offset and/or a stable portion of the QRS complex to time to peak dP/dt, or a stable portion of the pressure curve utilizing a pressure catheter or a filtered signal from the pressure trace or pressure sensor, as seen in FIG. 5a.

As seen in FIG. 5a, a heart may be provided with pacing electrodes 501 connected to pacing leads 502. A left ventricular pressure sensor catheter 503 may be provided through the aorta 504 to a left ventricular pressure sensor 505. In this way, a pressure catheter located within the left ventricle can be utilized to measure ventricular pressure and the derivative of the pressure waveform, as seen in FIG. 5a. The time from a reference (5), such as the onset of the QRS curve, until the LV pressure derivative curve dP/dt peaks (1) is measured, thereby giving a representation of onset of synergy, and also effectively a measure of time to peak dP/dt/QRS. Various other measurements are also shown in FIG. 5A, as well as how they may be displayed on a 3d heart model.

FIGS. 5b and 5c show an example of this determination of onset of synergy as measured from one animal study, which shows the onset of synergy when segment tension in the myocardium develops and stretching terminates. FIG. 5b show a model of the heart with schematic representation of sonomicrometry crystals 510 and epicardial sonomicrometric crystals 511 which are used to measure myocardial segment length trajectories in various positions in the heart, for example, as seen in the four different myocardial segment length trajectories 520 plotted in FIG. 5c. These are plotted together with the ECG trace and second order derivative of pressure for comparison purposes in FIG. 5c. It can be seen that the measured time reflecting time to onset of synergy, OoS, (i.e. the point at which segments are no longer stretching; where they have become stiff) reflects the peak in the second order derivative of pressure in the left ventricle. This is when the rate of change of pressure change in the left ventricle is at the maximum (i.e. a representation of the rapid increase in rate of pressure change), which results from the synchronous contraction of the myocardium.

A pressure curve can be compared with any pressure curve with the same time reference (5) to measure the time offset (2) between the curves or the different timing of two comparable curves with same reference, i.e. by calculating time delay 4 minus time delay 3. An example of such a comparison may be seen in FIG. 5d, wherein a reduction in time to peak dP/dt is seen with a different electrode position. Such a measurement may prove to be more robust than the non-invasive measures detailed above. Again, any measurement can be visualized on a surface of a heart geometry using color coded zones and a scale, relative to electrodes.

FIG. 5d also illustrates why known measures of mechanical activation are not suitable for determining synchrony, and the potential efficacy of any subsequent CRT. As can be seen, with pacing at both position 1 and position 2, the onset of mechanical activation occurs at a similar time point 51. However, the onset of synergy, i.e. the point at which the pressure begins to increase exponentially and where there is a rapid increase in the rate of pressure derivative (as seen in FIG. 5d), is significantly delayed in position 1, occurring only at time point 52, whereas this occurs soon after time point 51 in position 2. This rapid increase in the rate of pressure change reflects the point at which the pressure change begins to increase at a faster rate compared to that seen before, and occurs before the maximum value of pressure derivative. This point may be reflected in the final peak of the second order pressure derivative prior to maximum pressure, or aortic valve opening.

Such a delay may, for example, be due to dyssynchrony with isolated areas of the myocardium contracting, causing passive stretch of the myocardium, which is reflected in the comparatively low pressure increase. In this way, typical measures of mechanical activation, such as electromechanical delay (EMD) are measures of time of regional activation to onset shortening, only indicating the performance of the immediate area of myocardium. Further, in dyssynchronous hearts, EMD may vary within the heart, and this may also vary throughout the heart due to other issues, such as dyskinesia.

In contrast, onset of synergy is a global marker and reflects the phenomenon when active forces increase with global active or passive stiffening of segments (and any event that directly follows); a time when exponential pressure rise onsets (onset of myocardial synergy); a time at when any segmental contraction increases force and subsequently the pressure, without shortening segmental length (isometric contraction); once most segments are electric actively or passively stiffened. Mitral valve closure is typically an event resulting around the time of onset of myocardial synergy, and closure is a needed to allow a rapid pressure rise and isometric segmental contraction. Onset of myocardial synergy exist also in a situation when the mitral valve does not close, however, with incomplete closure of the mitral valve, segmental shortening will occur also after onset of synergy, and onset of synergy reflects in a rapid volume change of the left heart chamber rather than a rapid pressure increase.

Typically in the cardiac cycle one would name the electromechanical delay and the isovolumic contraction as the pre-ejection phase, and keep the EMD and IVC separate. IVC is characterized that there is contraction without shortening (i.e. that the volume is constant). In dyssynchrony there is a great overlap between EMD and isovolumic contraction, and during the isovolumic contraction period there is shortening and hence typically physiological characteristics of this period is lost. The pre-ejection period is therefore very different in a normal compared to a dyssynchronous heart, as is EMD and IVC.

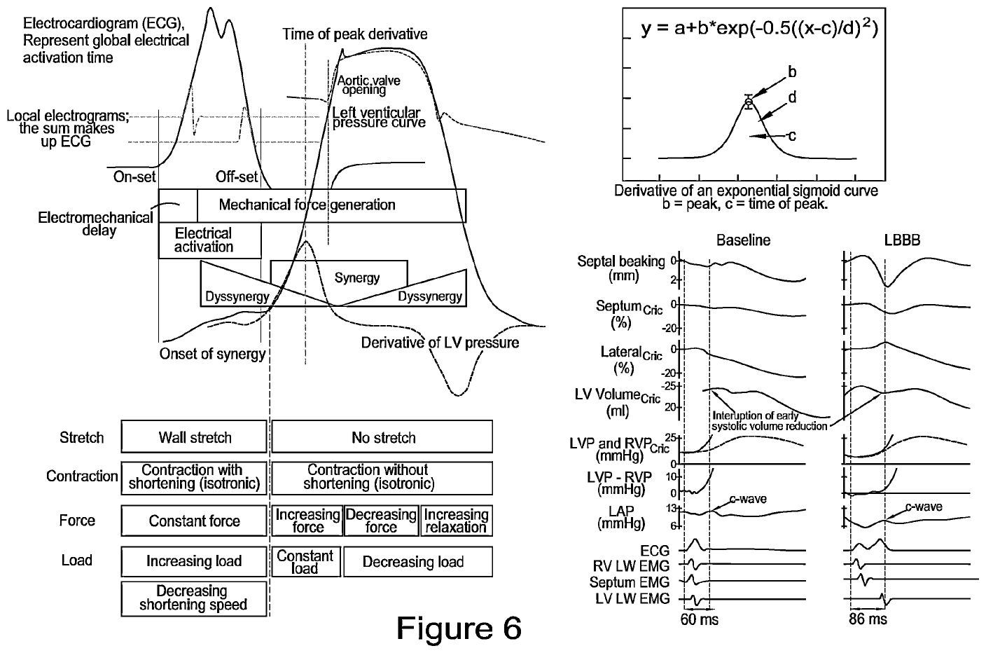

An illustration of physiological conditions experienced during heart contraction may be seen in FIG. 6. As is illustrated in this Figure, the onset of synergy is illustrated related to a representative ECG, showing the on-set and off-set of electrical depolarization of the heart represented in the QRS complex.

As described above, activation of the heart muscle requires electromechanical coupling. Electrical currents pass through the heart muscle within the specialized conduction system at high speed and within conductive muscle tissue at lower speed. With conduction block, in specialized tissue, propagation delays and becomes dyssynchronous with a pattern of conduction no longer determined by the specialized conductive tissue, but by the conductive properties in the heart tissue itself (muscle, connective tissue, fat and fibrous tissue).

Electrical activation is defined from the onset of an electrical stimulus that leads to depolarization of cardiac tissue (for example, as measured from the ECG curve or a pacing artefact) to the off-set of the QRS complex. An electromechanical delay is seen between the on-set of pacing and the beginning of local contraction (and also between local electrical and mechanical activation). However, as can be readily seen in FIG. 6, such a measure does not reflect the point at which the myocardium starts contracting as a global whole, thereby generating a rapid force. Rather, the early-activated muscle tissue starts contracting, however at no load, and hence shortens with minor force development and stretches relaxed or passive tissue to maintain the volume of the heart chamber. With more electrically activated tissue that shortens more relaxed or passive tissues are stretched, resulting in increased tension in stretched tissue and hence load. Once the electrical activation propagates throughout the heart, and more muscle shortens, there is no more tissue to stretch, relaxed or passive tissue have stiffened, shortening and dyssynergy stops and force develops with onset of synergy with exponential pressure increase until the aortic valve opens to allow muscle shortening again.

The onset of synergy relates to this point where the shortening of the muscle stops the myocardium contracts simultaneously, beginning to increase the force at a constant volume/load in the heart (a characteristic response seen with isometric myocardial contraction). This occurs at some point between the earliest, and latest regional EMD or later, and could be early or late in this phase, but rather reflects the degree of dyssynchrony. In itself, this point is difficult to measure, but this point is reflected in a number of measures, for example (but not limited to), early cardiac vibrations, pressure increase, peak derivative of pressure, aortic valve opening, aortic root vibrations, coronary sinus vibrations, filtered pressure waves, peak negative derivative of pressure. Such measures may have a constant relationship in time to the onset of synergy, such that the measurement of the time of such events will directly reflect the onset of synergy, and therefore may be used as a measure of onset of synergy. Therefore, by using such measurements to measure a representation of onset of synergy in time, it is possible to compare different pacing methods and their efficacy in reducing the time to onset of synergy. If shortening occurs when comparing to a different way of pacing, less dyssynchrony is present, and when the time delay gets longer more dyssynchrony is present.

Based on the results of the sensor measurements, it may also be possible to determine the most effective pacing regime to be applied. For example, a second circuit implemented in hardware and/or software may comprise an algorithm to determine how many electrodes should be included and in what position they should be placed in the pacing strategy, and further determines which pacing strategy to follow. For example, it may be determined that the most effective pacing may be achieved by CRT, His bundle, biventricular, multipoint or multisite, or endocardial pacing, or any combination of the mentioned in the form of a suggested algorithm of pacing. For example, if the onset of myocardial synergy with intrinsic activation is short, or if onset of myocardial synergy with optimal electrode positions gets longer, then physiologic/His pacing may be desirable.

A screen may be additionally provided for visualization of the heart model with any fiducials and representations of any sensor connected. Such a system may allow for an accurate measurement of cardiac dyssynchrony by the indirect measurement of the onset of myocardial synergy described above, such as by way of an accurate measurement of Time to peak dP/dt, time to zero crossing of a filtered pressure signal, time to peak Fc(t) based on CWT from acceleration or pressure signal, time to early vibrations in a time window of interest, and/or time to bioimpedance signal deviation. In this way, any shortening in the time to onset of myocardial synergy may be visualized with a corresponding shortening of any directly measured parameter as previously described, thereby indicating the presence of dyssynchrony. Equally, any pacing measures applied may be reversed when it is determined that dyssynchrony is not present. For example, when measuring the impedance phase and amplitude as an indirect measure of the onset of myocardial synergy in a case where dyssynchrony is not present, the impedance curves will not change with pacing at different locations because no change in contraction occurs with resynchronization.

As would be appreciated, certain limitations must be applied to the measurements to allow for meaningful data to be extracted from the measurements, and the measurements must be compared to a known time point. For example, it may be that measurements can only be performed during pacing if at least one of the following conditions apply:
1) That ventricular stimulation occurs before onset of QRS
2) That timing is corrected relative to onset of QRS
3) That the interval from atrial pacing to ventricular sensing (AP-RVs) is known.
4) A prolonged stimulus to QRS delay needs to be compensated

In order to provide effective pacing, any atrioventricular (AV) delay should preferably be calculated so that AP−VP is shorter than the shortest of AP−RVs and AP−QRS.

Preferably AP−VP should be calculated so as to equal 0.7*(AP*RVs), or if AP−QRS onset is known, the AV−delay interval should preferably be 0.8*(AP−QRS).

Measurements may be performed during ventricular pacing with intrinsic conduction, but only when the onset of the QRS complex is not ahead of pacing, unless the QRS onset−VP interval is corrected for in the measurement.

Measurements may be performed during atrial fibrillation with ventricular pacing when no fusion with intrinsic conduction is present. However, during atrial fibrillation pacing should preferably occur at a rate shorter than the shortest RR interval seen during a reasonable period in time so that when pacing occurs QRS complexes are not fused with intrinsic conduction, but are fully paced.

Measurements performed utilizing one sensor should only be compared with a similar sensor, unless a known correction factor is used to calibrate for differences between sensors. The detection of the reference in time should be similar, and carefully chosen to be the best representation possible of the similar time reference as compared with. A pacing stimulus may be initially negative, then positive in some configurations and equally may be initially positive, then negative in others. While the onset of the signal represents an unbiased reference in time that disregards polarity of the signal, then the maximum peak might be different in time between the two references, and the maximum should be compared to the minimum when this is the best possible detection for the signals with different polarity when compared. When intrinsic activation is detected, as in an intrinsic QRS complex, the onset of the QRS complex may be difficult to exactly define. In such a case, the earliest off-set from the isoelectric line should be chosen.

When the myocardium is paced (artificially stimulated), there is a delay from the pacing stimulus to the onset of activation such that there is a time delay from the onset of the pacing spike to the QRS onset. When comparing a measurement with a time reference from the QRS onset or the QRS complex with a measurement with a time reference from a pacing spike, such a time delay should be taken in account, for example by adding the same time delay to the non-paced measurement. The delay will typically be calculated based on the type of applied pacing. For example, the delay may be in the range of 10 to 20 ms. In typical disease, like a myocardial scar, pacing from within such a region may delay this interval beyond this range. Such a delay, typically beyond 20 ms up to 80 ms should be carefully analysed and compensated (either by pacing or by calculation) before utilized carefully for comparison.

In summary, when time reference or sensor is different between measurements, the off-set between the different time references or the sensors should be accounted for in the measurements for comparison.

In this way, it may be necessary to make sure, before measuring, that no activation occurs through the conduction system that would need to be compensated for in the measurement. The measurement of onset of synergy only takes meaning when one is not pacing the ventricle only for comparison with the surface ECG offset for determination of resynchronization potential as described.

By using the above described methods to measure the onset of synergy, it is possible to identify patients for potential CRT therapy. Traditional measures such as electromechanical activation and delay, onset of force generation, or local electromechanical delay cannot be utilized as suggested herein. As discussed, it is difficult to know exactly when to measure an electromechanical delay, as mechanical activation occurs over a wide range in time across the heart. Such issues can occur with all known methods of measuring electromechanical delay.

For example, should an isolated measure of electromechanical delay be measured using aortic valve opening, there would be many associated issues with such. In such a case, if one were to pace LV early, and allow intrinsic activation from RV, and measure from LV pace; then if pacing LV late, aortic valve opening would be determined by RV activation and not by LV, but the time from LV to aortic valve opening would be short. This gives a false measure of the efficacy of pacing in improving the physiological function of the heart.

Rather, by knowing the timing of activation through the normal conduction system, it is possible to compensate for measurements performed before pacing occurs. For example, if intrinsic activation occurs before pacing, then one should measure from onset of intrinsic and add the interval from pacing to activation, to allow comparison with other measurements when pacing.

### Filtered Traces for Determination of Onset of Synergy

It has been further found by the inventors that the signature of the cardiac phases lies in the frequency spectrum after the 2^{nd} harmonic of the left ventricular pressure trace, where the harmonic is represented by 1/paced cyclerate (s). Early contractions at low pressures (i.e. the contractions that are associated with dyssynergy) do not produce high-frequency pressure components. However, the rapid increase of pressure that occurs with onset of synergy results in high-frequency components of the LVP trace. In this way, the crossing of the x-axis at zero for the 2^{nd} and above harmonics captures only the synergy components, and can therefore be used as a reference measure to compare with QRS onset or onset of pacing. Similarly, dyssynergy (being characterised in early contractions) does not produce high-frequency components.

With the onset of contraction load against initial load (LO), contraction velocity rapidly increases (Vmax). With contraction, the load increases to Lmax, at the point where V goes to 0. Tension follows a sinus wave, and with synergy tension increases above the sinus envelope.

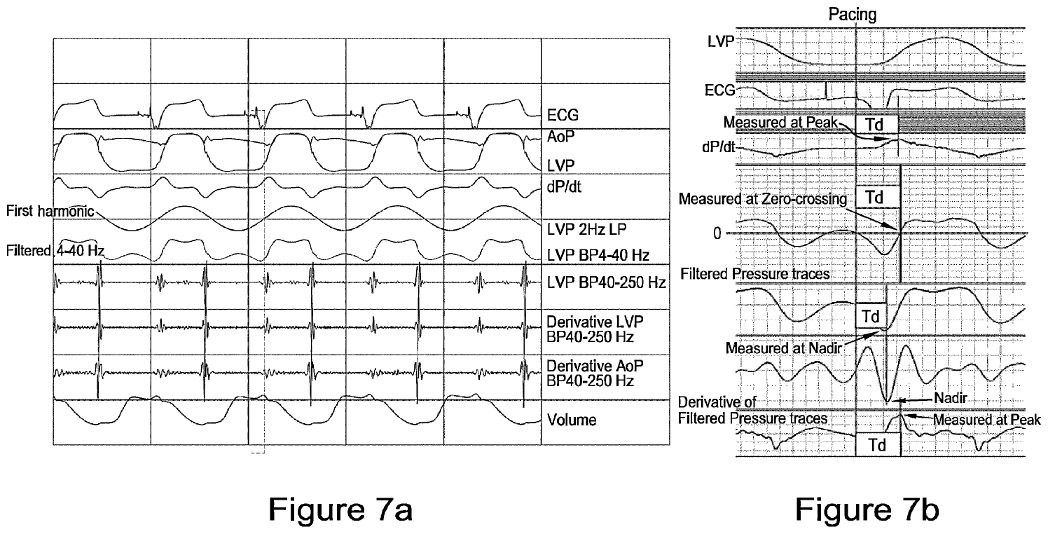

As can be seen in FIG. 7a, filtering of the LVP demonstrates an underlying basal sinus wave in the first harmonic that reflects the heart rate. The following 2^{nd} and above harmonics contain the information that shapes the sinus wave into a characteristic pressure waveform. High frequency (for example 40-250 Hz) components initiates with onset of contraction and mid range frequencies (for example 4-40 Hz) increase from onset of synergy until aortic valve opening. The inventors have discovered that, when the above mentioned filtered pressure range crosses 0 it is timely connected to peak dP/dt, and to onset of synergy, and therefore may be representative of the onset of synergy. Synergy with increasing force and exponential pressure increase above the sinus waveform starts with onset of synergy and stops with aortic valve opening.

High-frequency components can be assessed as vibrations and translate from the left ventricle to the aorta and surrounding tissue through the solid fluids and tissue. Filtering high pressure components from aortic pressure (AoP) waveforms or atrial pressure waveforms or coronary sinus waveforms, or detecting vibrations using accelerometers or any other sensor will therefore reflect synergy, and as long as the measurement occurs at a similar position on the measured trace/curve, for example, when the trace crosses zero, from the onset of vibrations or a certain characteristic of a waveform, or a template waveform. Such high frequency components (for example, those above 40 Hz) may additionally find use in improving the identifying of onset of synergy in the mid range filtered signal (such as a 4-40 Hz) signal, as the high frequency components identifies the onset of pressure rise prior to zero-crossing.

FIG. 7b shows various other traces from various filtered waveforms, and how they may be used to give various measures of Td, each of which relates to the onset of myocardial synergy, OoS. By taking one of these measures, and measuring how it varies with pacing, then it is possible to identify the presence of dyssynchrony in a patient due to the constant delay between the specific measure of Td and the actual event of onset of myocardial synergy.

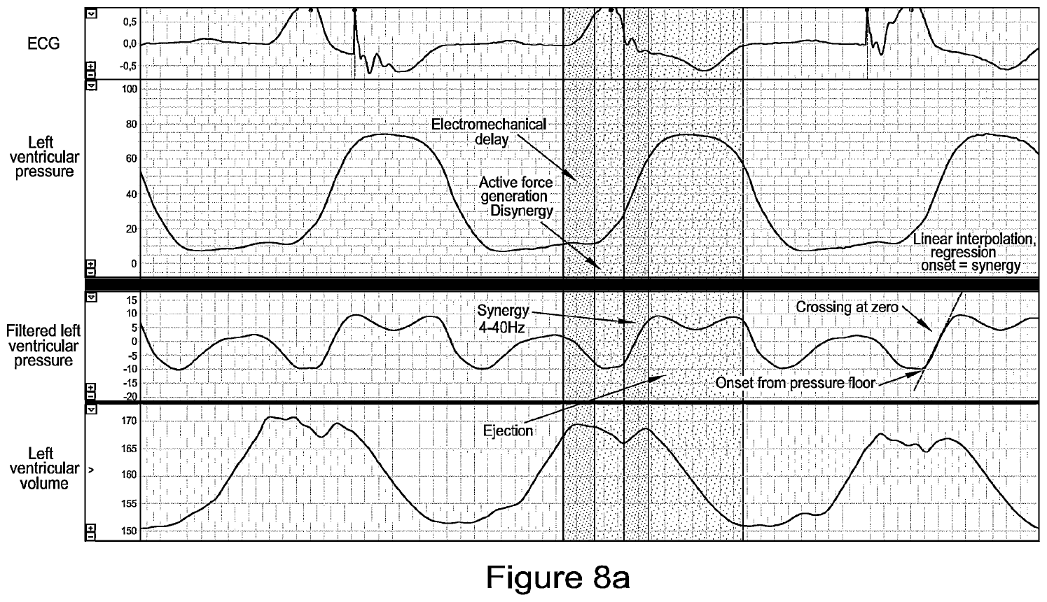

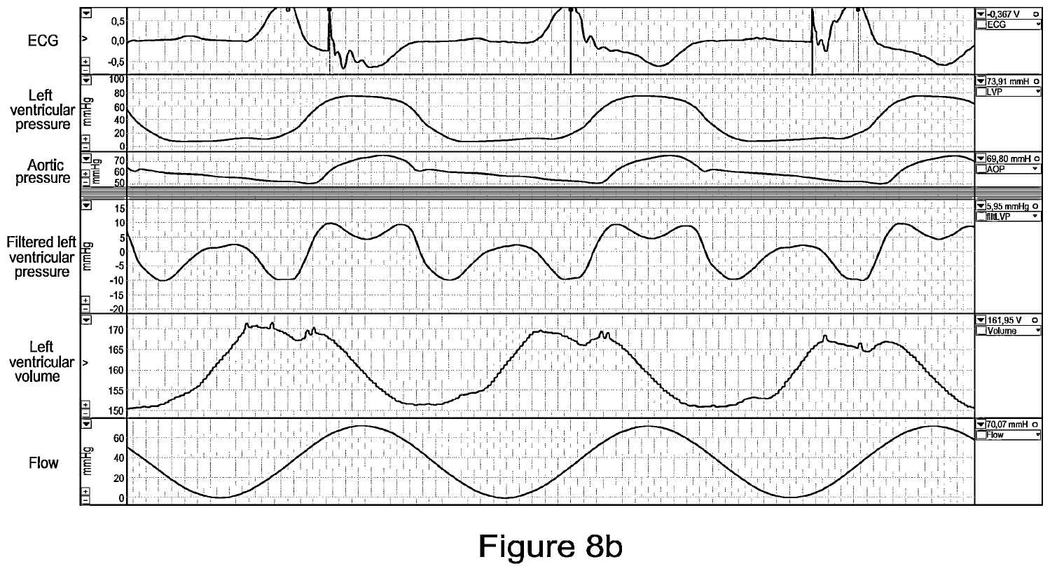

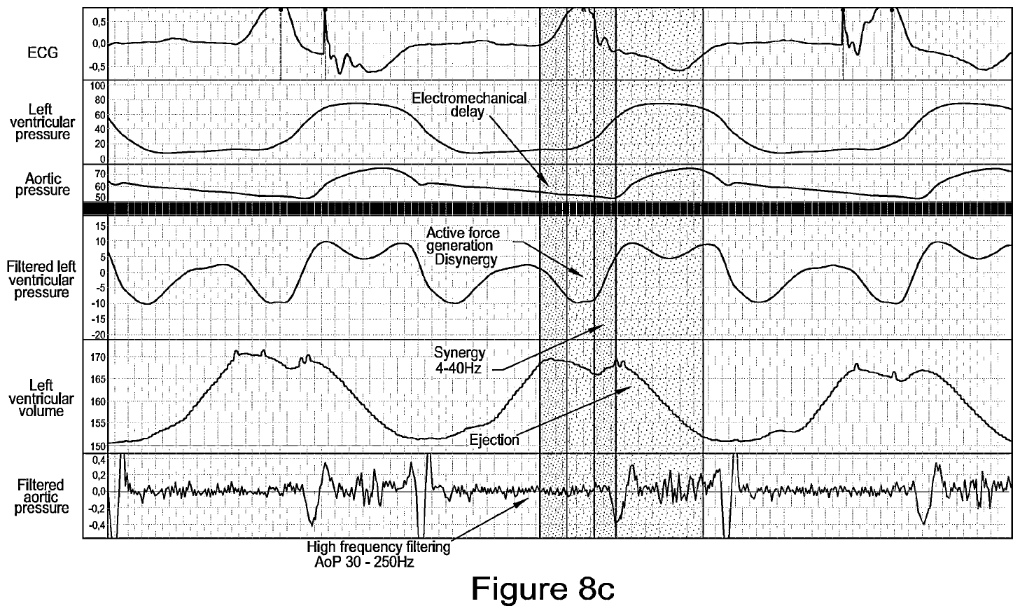

Further information regarding the onset of synergy may be deduced from filtering various measured signals, as seen in FIGS. 8a, 8b and 8c.

Starting from FIG. 8a, each phase discussed above is annotated on the traces. Initially, there is a delay between the onset of pacing seen on the ECG trace, and the beginning of increase in LV pressure.

Then, there is dyssynergy when the mechanical force begins to slowly increase, due to the passive stretch of the myocardium. Low-frequency components in left ventricular pressure (less than 2^{nd} to 4^{th} harmonics of the heart rate) are typical for dyssynergy. With dyssynergy there is onset of active force with sarcomeric cross-bridge formation at high rate in specific regions of the heart that result in shortening of the sarcomers (and myofibrills) that leads to stretch of not yet contracting segments and regions of the heart, with only a small increase in pressure resulting (with low-frequency components), as discussed extensively above.

The onset of synergy is reflected in a rapid increase of force at a relatively constant volume, which is reflected in the increased rate of increase of pressure. With activation of all segments and synergy, pressure increases rapidly (with high-frequency components) when approaching isometric (and isovolumic) conditions as load increases. This can, for example, be seen in the identifiable change in the rate of increase of the left ventricular pressure between the initial (relatively) slower increase in pressure due to dyssynergistic contraction and the exponential increase of the synergistic contraction. This may be seen in a step change in the rate of increase of the left ventricular pressure, and/or may be identified by further post-processing of the data. For example, this change can be measured in the frequency range, as the frequencies contained in the pressure trace increase when there is a step change in the pressure change. This occurs beyond the low order harmonics of the frequency spectrum, and the OoS may become evident when low order harmonics are filtered with a low pass filter or band pass filter. Filtering at, for example, a band-pass 2-40 Hz or 4-40 Hz removes the low, slow frequencies that are associated with dyssynergy and the onset of synergy may be seen as the onset of the pressure increase that leads to, or is directly prior to aortic valve opening or maximum pressure. Alternatively or additionally, this may be seen in the peak second order derivative of pressure rise in the left ventricle. Filtering can be adaptive applying harmonics relative to the paced heart rate or any other adaptive filtering technique.

This change in rate of pressure increase is because of increasing and exponential cross-bridge formation while passive stretched segments tension increase, either because depolarization or because elasticity model reaches its near maximum. Rapid cross bridge formation with isometric or eccentric contraction leads to high-frequency components in the pressure curve frequency spectrum, reflecting onset of synergy. This phase of the cardiac cycle may be seen when filtering LVP with high pass filter above the 1^{st} or 2^{nd} harmonics. The filtered and characteristic waveform has a near linear increase, from onset of synergy to crossing 0, and continues with a linear increase up to aortic valve opening. The line of linear increase reflects the period with synergy, crossing zero at halfway in the phase, which corresponds to peak dP/dt as described above, and onset of synergy is reflected in where this line starts to rise above the floor of the filtered pressure curve or at its nadir.

Ejection then occurs with the opening of the aortic valve, thereby reducing the LV volume at a relatively constant pressure. Another example trace is seen in FIG. 8b, which has been annotated to show each of the above phases in FIG. 8c. FIG. 8c also shows a high-frequency filter of the aortic pressure, which also shows peaks in the high-frequency domain at points that could be used as a measure of OoS (onset of synergy).

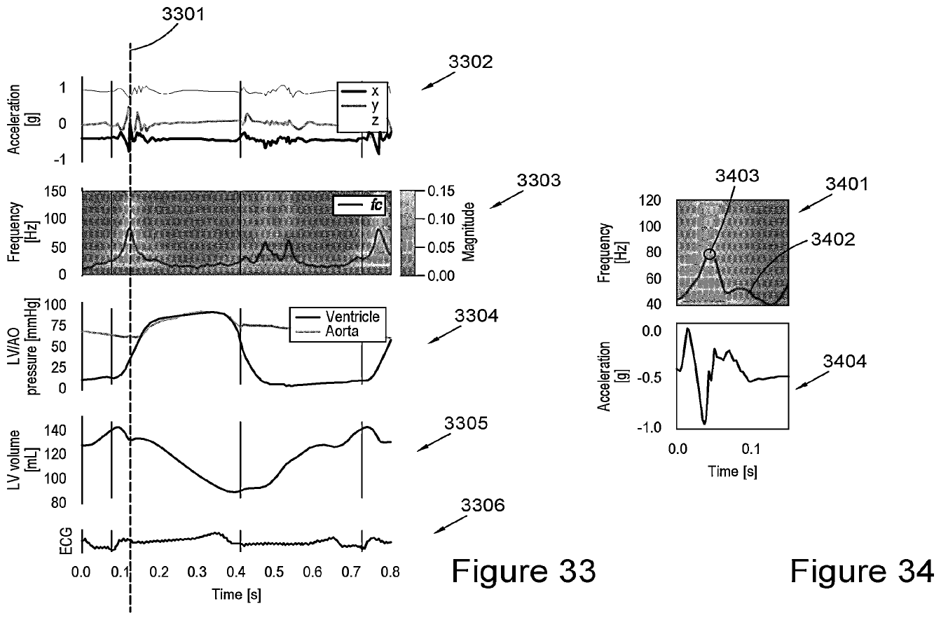

Other data may alternatively or additionally be analysed in order to determine a measure of the onset of synergy. In this way, other measures may be used either as a supplement to measuring pressure traces, and determining therefrom the time of onset of synergy (or an event related thereto) as considered above, or as an alternative to pressure traces. For example, acceleration data may be analysed, such as that provided by an accelerometer sensor, as is illustrated in FIGS. 33 to 35.

FIG. 33 shows various traces that can be extracted from accelerometer data. Graph 3302 shows raw acceleration, from which a wavelet scalogram 3303 may be produced, which shows the frequency spectrum over time. Graph 3304 shows the left ventricular pressure (LVP) and the aortic pressure (AOP), graph 3305 shows LV volume, and graph 3306 shows a detected ECG. FIG. 34 shows a zoomed in extract 3404 of the bottom trace of the acceleration of graph 3302, and a zoomed in extract 3401 of the wavelet scalogram of graph 3303. From the wavelet scalogram, a trace 3402 may be derived which represents the center frequency for each time point. It has been discovered that the peak of this frequency 3401 within a given time frame accurately represents the time of the onset of synergy. This may be plotted as point 3301 against several traces, as shown in FIG. 33. Whilst FIG. 34 shows only a single axis of acceleration (in this case, the x-axis acceleration) it would be appreciated that a similar analysis could be performed for all axes, and only a single axis is illustrated for clarity purposes.

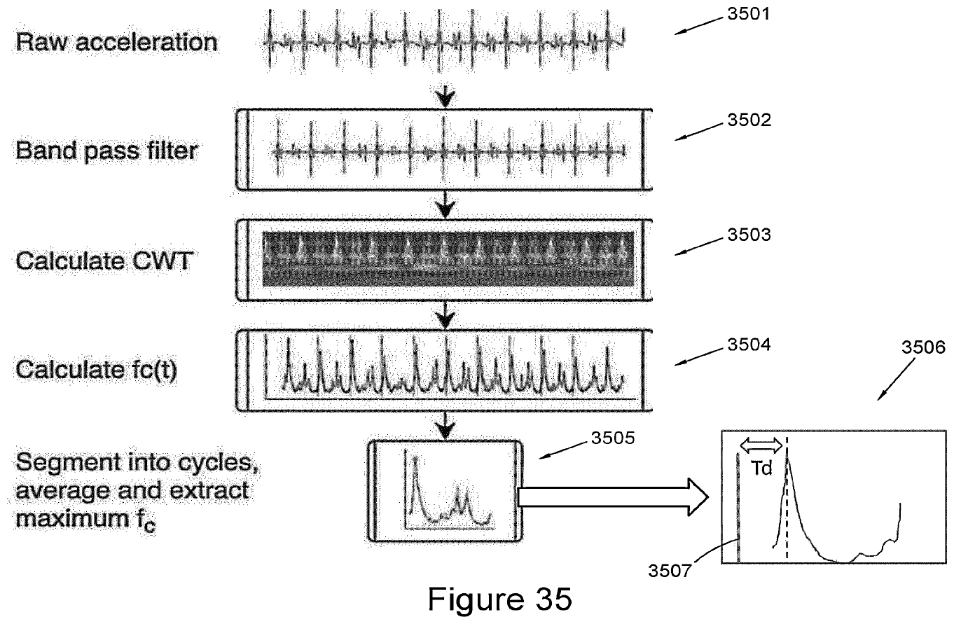

FIG. 35 shows an example analysis that may be performed to acceleration data, so as to calculate a time to onset of synergy. For each axis, raw acceleration is measured. A plot of the data from one axis of raw acceleration against time may be seen in graph 3501. The raw acceleration data then may be band pass filtered, resulting in the data seen in graph 3502. From such a band-pass filtered dataset, the continuous wavelet transform CWT) may be calculated, resulting in graph 3503. The center frequency trace fc(t) is then calculated from the CWT as seen in graph 3504. By splitting the fc(t) trace into cycles 3505 corresponding to the heartbeat, averaging each cycle and extracting the time of the peak fc(t), it is possible to determine the time-to-onset of synergy (Td) as seen in graph 3506. The time to onset of synergy may be measured from any suitable reference time, such as the QRS-onset, 3507.

As would be appreciated, acceleration data may be used as a standalone measure. or alternatively, it may be used in combination with other measures such as the pressure traces, and/or filtered pressure traces so as to determine the time until the onset of synergy.

### Further Discussion of the Onset of Synergy

As would be appreciated from the above (and following) description, the point of the onset of synergy may be determined in a number of ways, essentially by detecting the point (or a point directly related to) the time during cardiac activation where the myofibrills work in synergy and begins to contract isometrically as most of the myocardium stiffen from either active contraction or passive stress (increased resting tension), which results in an exponential pressure increase (rapid pressure rise) within the heart. The following example methods are not intended as an exhaustive list of ways in which the point of onset of synergy can be measured, and utilized, but are rather presented as examples to illustrate the present invention.

When it is possible to determine the point of the onset of synergy, and how it changes with various types of treatment (for example, with intrinsic rhythm, RV pacing, LV pacing and/or BIVP amongst others), it is possible to identify whether the concept of synergy exists within a patient. Where it is identified that the time to onset of synergy can be shortened, then it may be said that “synergy” exists for a determined pacing regime, and therefore that a patient may benefit from treatment.

It is important to note that, as would be understood by the skilled person, the methods presented herein do not require the presence of a patient, nor do they explicitly require the collection of data from the patient. Whilst patient data is required, the measurements may be (and typically are) performed after the collection of data, and away from the patient. It is therefore envisaged that the inventions described herein may be performed on pre-existing data sets, without the presence of a patient. In this way, an examination of a patient involving the collection of data is not integral to the present invention. Any reference herein to steps that involve the collection of data would be understood such that they refer to steps and measurements that have already been performed. In this way, the methods herein may be considered as methods of processing such data so as to give technical information regarding the patient, which may then be used for in planning how best to give/improve the prognosis of the patient from whom the data was previously collected.

Cardiac Resynchronization Therapy (CRT) is understood, and can be achieved in multiple ways either by direct stimulation of the conduction system of the heart chambers (left bundle branch or His bundle) or with stimulation at more than one site (resynchronization therapy). CRT can be permanently applied with a pacemaker or temporarily with electrophysiology catheters or pacing leads to perform artificial stimulation of the myocardium. CRT also implies that there is an intention to perform resynchronization with any kind of artificial stimulation of the heart chamber or chambers. One may also consider intrinsic conduction in a patient as resynchronization, and compare the intrinsic activation to an artificially paced beat or an ectopic intrinsic beat in the heart of the patient.

The calculation of the time of the onset of synergy may be utilized as a prognostic biomarker, in that if a patient (after having Cardiac Resynchronization Therapy) has a late onset of synergy during stimulation (with CRT or pacing electrodes), then the prognosis of the patient will be poor. In this way, it could be said that there is described a method to determine the prognostic results from resynchronization therapy, from data that has been obtained from a subject when controlling their heart rate and sensing the ventricle, either by stimulation of the atrium or by sensing the atrial electrical activity while sensing the ventricle. Then CRT is applied and the signals from sensing electrodes and sensors are collected. Measurements of the intervals and comparison of the data is performed in a processor outside of the body after collection of the data to determine if the pacing pulses have provided synergy or not. The finding of an improvement in synergy is present when a first interval is shorter than another interval. If with CRT, synergy is present, then the prognosis is determined to be good.

As described above, it may be desirable that, for an accurate measure of the onset of synergy, it is ensured that the electrical activation and resulting pressure increase in a data set results only from the stimulated sites and not from the intrinsic activation of the heart. Therefore, in combination of the methods that are considered herein or alone, pacing electrodes may have been placed in the atrium and ventricle(s), and pacing may be applied from the atrium and/or, for example if atrial fibrillation is present, then from the ventricle, both pacing being at rates 10% above the intrinsic heart rate. Therefore, from data received during pacing at a higher rate that the intrinsic activation, an automatic detection of a set of intervals may occur, for example:
Detecting of the atrial paced to the surface ECG onset and offset
Detecting the atrial paced to the right ventricular sensed interval
Detecting the atrial to the left ventricular sensed interval

In order to provide a fixed interval until the chambers are activated, and ensure that intrinsic activation does not interfere with the measured response, there may be pacing with a paced atrial to paced ventricular interval at 40% shorter than any of the detected intervals. This ensures that the chambers are not activated by intrinsic activation, and therefore that the paced activation and the intrinsic activation are not competing, which can lead to an inaccurate measure of the time of the onset of synergy.

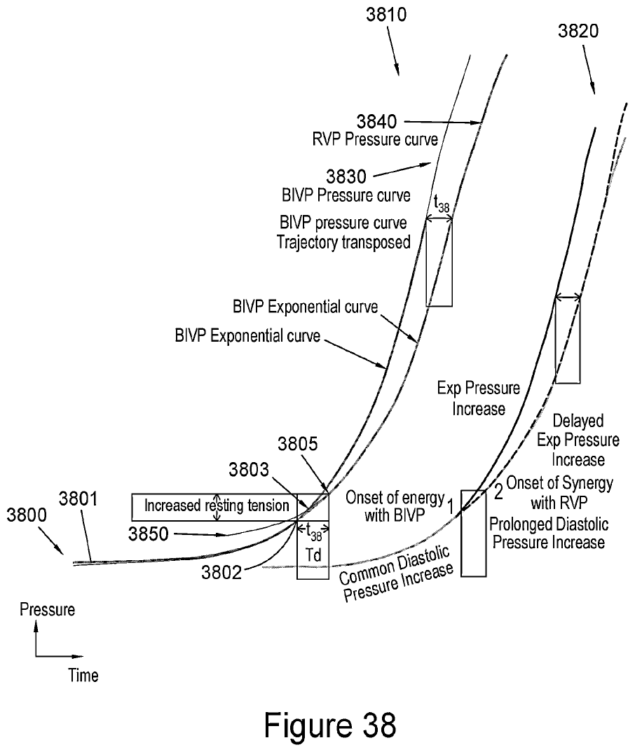

The measurements above relating to the identification of the onset of synergy may be utilized in various different ways to give an indication of whether pacing results in (an increase of) synergy. Other ways of illustrating and/or measuring the point of the onset of synergy are envisaged, such as that of FIG. 38. The onset of synergy results in a repeatable pressure increase that follows a trajectory over time up until peak dP/dt that can either be represented as a template (as in FIG. 38) or an equation. By comparing the pressure curve before and after CRT, and shifting the resulting curves (with/without CRT) such that the pressure curves then track each other, it is possible to determine the delay to the onset of synergy, by the amount it was necessary to shift the curves so as to match each other. This time delay remains constant throughout the pressure curves.

For example, FIG. 38 shows a comparison 3810 over time between a pressure curve that results the pacing the right ventricle 3840, and from bi ventricular pacing 3830. As can be seen, from point 3800. The curves for RVP 3830 and BIVP 3840 are parallel, and are both aligned by a time point 3801 that is a common point of atrial stimulation in both responses. Then, the measurement of a subsequent pressure rise follows. Said another way, although the curves for RVP and BIVP relate to different heart beats, they are fitted together relative to their stimulation timing, and the pressure level is adjusted to fit the diastolic portion of the curves prior to ventricular pacing.

From comparing these curves, whether synergy is present (i.e. whether the time to the onset of synergy has been shortened by providing BIVP), and the timing of the onset of synergy may be measured by finding the point of deviation between the fitted pressure curves that are compared.

As can be seen in FIG. 38, and specifically in the comparison 3810, whilst the RVP pressure curve 3840 and BIVP pressure curve 3830 are parallel (follow the same trajectory) to begin with, they begin to deviate from point 3802, which represents the time of the onset of synergy with BIVP.

The inventors have recognized that, despite the difference in timing of the onset of synergy, the pressure rise preceding the onset of synergy will follow a common diastolic pressure increase, and then the pressure rise resulting from the onset of synergy will always have the same shape (i.e. follow the same mathematical equation on a plot between pressure and time beginning from the onset of synergy), despite the delay, and change between the relative resting tension. Therefore, from the determination of this point, it is possible to fit this portion of the pressure curve resulting from BIVP onto the corresponding portion of the pressure curve resulting from RVP. From this, it is possible to use the amount that it has shifted in order to determine pertinent information about how BIVP has changed the onset of synergy, and thereby determine whether synergy is present with such a method of pacing.

For example, as shown in FIG. 38, a portion of the BIVP pressure curve 3830 can then be fitted onto the corresponding curve relating to RVP that follows the point 3802 where the BIVP and RVP pressure curve deviate (which is denoted by an arrow on the BIVP pressure curve 3830). This shifted BIVP pressure curve 3850 crosses the original BIVP pressure curve 3830 at point 3803, which indicates the onset of exponential pressure rise. Points 3802 (i.e. the onset of synergy) and 3803 (the resulting onset of exponential pressure rise) are the points that mark the timing of deviation between the RVP pressure curve 3840 and BIVP pressure curve 3830. In the example of FIG. 38, the BIVP pressure curve 3830 is shifted up and to the right to shifted pressure curve 3850, such that the portion of the BIVP pressure curve following point 3802 (up until peak dP/dt), to the point where the shifted pressure curve 3850 matches the RVP pressure curve 3840. The portion of the BIVP pressure curve following the onset of synergy 3802 fits on the RVP pressure curve starting at point 3805, and from this point follows the same curve as the RVP pressure curve. Therefore, as stated above, as the increase in pressure following the onset of synergy in the same patient follows the same pressure rise until peak dP/dt, it may be said that the onset of synergy in the RVP pressure curve occurs at point 3805.

By comparing the difference in the onset of synergy during BIVP (at point 3802), and the onset of synergy during RVP (at point 3805), it is possible to obtain valuable information regarding how the change in pacing effects the function of the heart. The time delay (t 38 in the example of FIG. 38) can be used to show that BIVP results in a shortening of the time to onset of synergy in a patient, thereby indicating how a pacemaker may be programmed to improve the prognosis of a patient. Additionally, the vertical offset between points 3803 and 3805 shows the increase in resting tension in the myocardium that results from dyssynchronous contraction of the ventricles, and passive stretching of the heart muscle in advance of the onset of synergy.

FIG. 38 also shows comparison 3820, which is a simplified version of comparison 3810. This shows the common diastolic pressure increase between BIVP and RVP, and then the point of deviation 1 (i.e. the onset of synergy with BIVP) leading to exponential pressure increase with BIVP. The portion of the BIVP curve following point 1 may be fitted onto the corresponding portion of the RVP pressure curve, indicating the onset of synergy with RVP at point 2, which results in the (comparatively) delayed onset of synergy with RVP. This time delay remains constant throughout the BIVP and RVP pressure curves.

As would be appreciated by the skilled person, this process can be automated and for the data resulting from any number of pacing regimes, whether by a simple matching of the curves (for example, by the fitting of a template to the pressure trajectory with a least squares method) or by a comparison of the mathematical formulae that represent the curves. There can be an automatic detection in the data of the exponential pressure rise, up to the peak dP/dt which results from the onset of synergy. From this, there may be an automatic calculation of the exponential formula that fits the pressure curve, and from this, the time when the exponential formula fits one of a number of curves can be determined. For example, there could be a template match, and there be calculated a time offset between the exponential formula and the template matches, or equally a cross-correlation between other measures. Additionally, whilst this is shown in the example of FIG. 38 with regards to the raw pressure data that can be obtained from the heart, it would be appreciated that these measures are reflected in all pressure measurements, including filtered pressure measurements. For example, as there exists a common mathematical equation that can describe the pressure rise that results from the onset of synergy for a given patient, the time delay to the peak dP/dt following various kinds of pacing can be compared so as to give an accurate representation of how pacing affects the time delay to the onset of synergy, and therefore be used to advised on a suitable pacing method and a programming of a pacemaker for the most effective treatment.

From the above, an output of the time to onset of synergy and the offset between exponential pressure rise curves, or offset between band pass filtered curves, or between derivatives of pressure curves can be provided. If the onset of synergy is shorter than just in RV pacing then it might be decided that it would be beneficial to program an implanted pacemaker to pace from both RV and LV channels. Equally, it might be recommended to modify pacing so as to occur with multiple channels, and the delay to onset of synergy is shorter with any multipoint/multisite pacing, then it might be suggested to program the pacemaker to pace in a multipoint/multisite way.

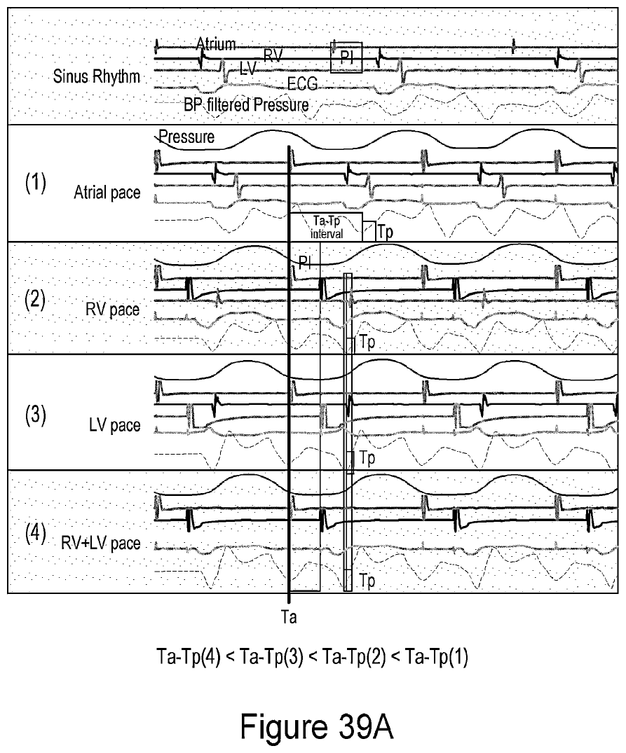

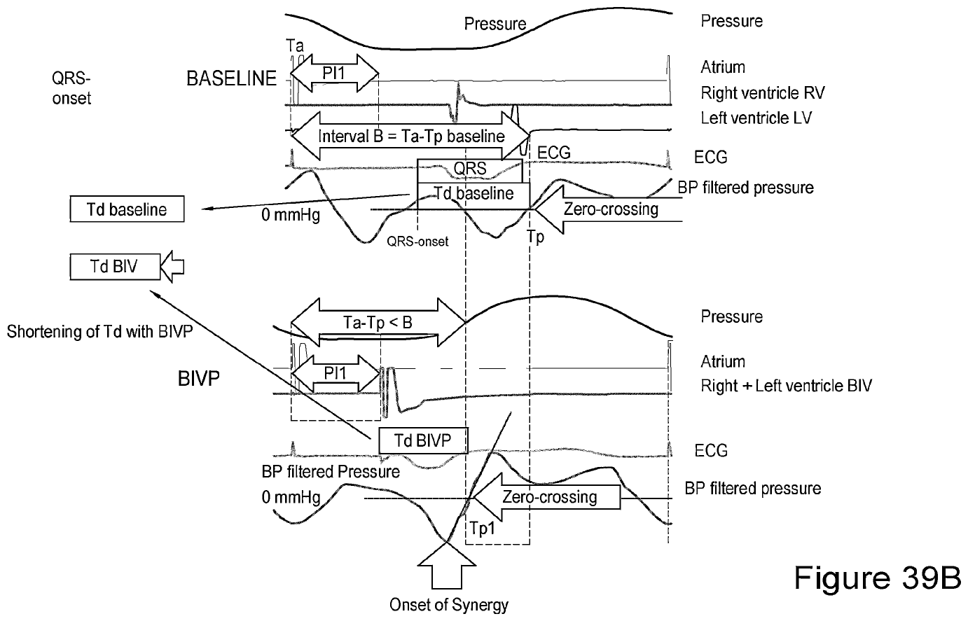

FIGS. 39a and 39b show another way in which an advancement in the onset of synergy can be detected, specifically by an advancement of the zero-crossing of the filtered (band-pass) pressure curve (Tp) with stimulation from both LV and RV compared to when either LV or RV is paced, and therefore in this example, it may be said that synergy is present and therefore that it may be desirable to undergo CRT using BIVP. FIG. 39b shows a more detailed view of the traces of FIG. 39a, for clarity and ease of reference.

FIG. 39a displays traces that have been gathered in 5 separate cases, one with the natural sinus rhythm, on with atrial pacing, one with RV pacing, one with LV pacing, and one with RV and LV pacing (BIVP).

As discussed above, synergy is the phenomenon by which stimulation by a given pacing regime leads to a sooner onset of synergy. This may be identified by the advancement of rapid pressure rise, which can be identified by a leftward shift in the zero-crossing of the band pass filtered pressure curve. The onset of synergy (OoS) is the corresponding onset of pressure rise along the tangent of zero, as can be seen in FIG. 39b. Therefore, a leftward shift in the zero-crossing of the BP filtered pressure curve is directly related to the OoS, and therefore can be said to correspond to a leftward shift in the OoS.

The OoS can be compared to the rapid pressure rise with pacing or with intrinsic rhythm from onset of electrical activation, and if OoS is advanced when compared to the other then it may be said that more synergy is present. FIG. 39b shows that, with BIVP Ta-Tp is shorter then Ta-Tp at baseline, confirming that Td is not a result of intrinsic conduction. Td measures the time from electrical activation to Tp and is a referenced interval of the time to OoS. As can be seen in FIG. 39b Td is shorter with BIVP compared to Baseline, synergy is present with BIVP and it may be desirable to undergo CRT using BIVP.

In order to populate the traces of FIGS. 39a and 39b, data related to the OoS may be collected from a pressure sensor in the left heart chamber and subsequently analysed, relative to various timings of the heart that are collected from electrodes placed within the atrium and/or right ventricle and left ventricle, as well as surface electrodes that have collected a corresponding ECG signal.

As above, the pressure signal can be band pass filtered at 4-40 Hz to remove the high and low frequency waves, and simplify the subsequent analysis. The corresponding ECG signal, to which the pressure signal is aligned and compared.

The ECG signal is passed on to a processor unit, and a time of atrial intrinsic activation/stimulation (Ta) can be determined. The signal from the pressure sensor is also provided to a processor unit, where the value of 0 can be determined from the BP-filtered pressure waveform, and the time may be extracted (thereby giving a measure of Tp). From this, a baseline interval B can be calculated, as equal to Ta-Tp (i.e. the time between activation and zero crossing of the pressure curve for intrinsic activation). Intervals PI, Ta-Tp, Td and QRS-onset are demonstrated in FIG. 39b.

Then, following pacing of the heart chamber from a first electrode (for example, one of the electrodes positioned in the RV or LV) at a set pacing interval (PI1) after Ta (but before QRS-onset), a corresponding Tp1 may be calculated. The value of 0 is determined from the BP-filtered pressure waveform and the time is extracted (Tp1).

The pacing interval (PI1) is reduced, typically to more than 20 ms before QRS-onset, until the corresponding interval Ta-Tp (Ta-Tp1) is less than B (the baseline interval between activation and zero crossing of the pressure curve for intrinsic activation). For example, the pacing interval that results in a Ta-Tp<B is PI1, and the corresponding Ta-Tp interval (Ta-Tp1) at PI1 equals T1.

Then, pacing of the heart chamber is performed from a second electrode (i.e. another one of the electrodes) at a set pacing interval (PI2) after Ta, and the corresponding Tp2 is registered. In this way, the zero-crossing is collected from the corresponding BP-filtered pressure waveform and the time is extracted (Tp2). Again, the pacing interval (PI2) is reduced until the corresponding interval Ta-Tp (Ta-Tp2) is less than B, which is again typically more than 20 ms. For example, the pacing interval that results in a Ta-Tp<B is PI2, and the Ta-Tp (Ta-Tp2) interval at PI2 equals T2.

Then, pacing of the heart chamber is performed from multiple electrodes (for example, both the RV and LV electrodes) relative to Ta at a set P13, which corresponds to the lower of PI1 and PI2. Then T1 and T2 is repeated with P13 with stimulation at each electrode, the value of 0 is collected from the BP-filtered pressure waveform and the time is extracted for T1 and T2. Then stimulation of combined electrodes with P13 and the corresponding interval Ta-Tp (Ta-Tp3) is registered. The resulting Ta-Tp (Ta-Tp3) interval at PI3 equals T3, and it may be said that synergy is present if T3 is lower than T1 and T2 at P13. If this is the case, then it is desirable to perform synergistic pacing from multiple electrodes in CRT. Conversely, if T3 is higher than T1 or T2 then synergy is not present and synergistic stimulation cannot be performed. Following a positive determination for BIVP, a pacemaker can be programmed with corresponding intervals for P13 relative to Ta for synergistic stimulation of the heart. The steps can be repeated with different electrode positions to find the shortest interval T3 compared to T3 from different electrode positions.

Finally, a Tbaseline can be calculated by measuring the interval from QRS-onset to Tp and adding 15 ms+P13. Td BIV equals removing the interval P13 from T3, and Td baseline equals removing the interval P13 from Tbaseline. It may be said that synergy is present if T3 is lower than T baseline (i.e. that the time to Td has been shortened when comparing between pacing, and intrinsic conduction). In sum, when calculating Td it may be said that synergy is present when Td BIV is lower than Td baseline. When pacing the specialized conduction system with only one electrode (T2 and P13), it can be said that synergy is present if T2 is lower than Tbaseline.

Similar data may be employed for synergistic pacing from different PIs from a pacemaker. In such a method, a pacemaker is programmed with corresponding intervals for PI1 for the first electrode, and PI2 for the second electrode to provide synergistic pacing to the heart. Each PI must result in a corresponding Ta-Tp shorter than B. The value of 0 is collected from the BP filtered pressure waveform and the time is extracted (Tp1). The onset of the QRS complex is identified and time is extracted (Tqrs) at baseline and with each pacing. Td baseline is the Tqrs to Tp interval with intrinsic activation without pacing the heart chamber. Td for the pacing electrodes and PIs equals the time interval from Tqrs to Tp1. Then a new P13 is added for any of the electrodes or a new electrode and pacing is provided from two or more electrodes, a new Tp2 and corresponding Td (Tqrs to Tp2) is calculated. Again, a lower Td indicates that more synergy is present with the corresponding PIs. If Td with pacing (BIVP) multiple electrodes and PIs is lower than Td baseline then Synergy is said to be present with pacing and the pacemaker can be programmed to stimulate the heart at the corresponding electrodes with the corresponding PIs. If synergy is present, then the pacemaker can be programmed to stimulate at the two electrodes. As would be readily appreciated by the skilled person, further, additional electrodes and PIs can be added and stimulated simultaneously, or with a delay between the electrodes (configurations). Typical delays (PIs) are between 10-60 ms.

In such a case, various configurations may be noted. A configuration that shortens Td below all other time intervals is noted as improved synergy, and therefore the pacemaker can be programmed to stimulate electrodes with the applied configuration that results in the soonest/earliest Onset of Synergy.

Such a method may similarly be performed with the detection of synergy from the pressure curves, as described more fully above with regards to FIG. 38. If two electrodes are simultaneously, or with a delay, stimulated, then the earliest identifiable part of the unchanged pressure curve (for example, above 80% template match) (including nadir, O-cross, template, min max), should be noted and compared to stimulation and configuration from any other electrode pairs. If a pair of electrodes stimulated with a configuration advances a part of the curve compared to the others, then synergy is present with such a configuration, and the earliest part of the curve that is advanced is onset of synergy and is the time point to which measurements are performed. A pacemaker can be programmed to perform stimulation at the point of electrode positions and configuration.

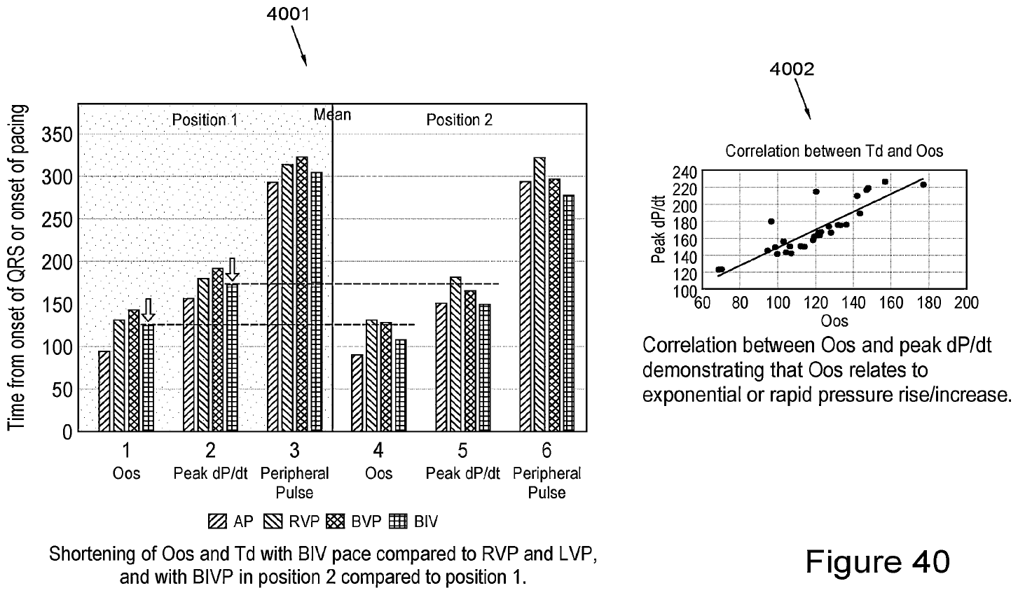

FIG. 40 shows two graphs, 4001 and 4002. Graph 4001 shows the shortening of OoS (measured as nadir of the BP filtered pressure curve) and Td (time to peak dP/dt, which is shown by the zero crossing of the bandpass filtered pressure curve) with various kinds of pacing in various positions. In this case, it may be said that the time to OoS is reduced further with pacing from position 2, and therefore it may be desirable to provide pacing from position 2. Graph 4002 shows the correlation between OoS and peak dP/dt, demonstrating that OoS relates to the peak exponential pressure rise within the heart, and as noted in FIG. 38, that the delay that result from a delayed onset of synergy is constant up to peak exponential pressure rise.

Summary of Onset of Synergy

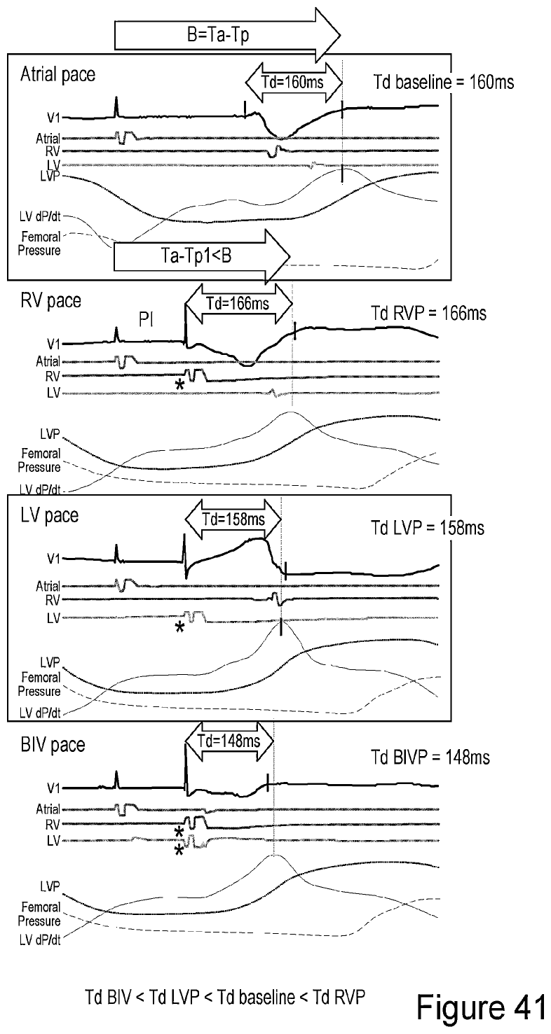

Essentially, the inventor in this case has discovered a new measure that can be used to effectively identify patients that are suitable candidates for cardiac resynchronisation therapy, by measuring the point, termed onset of synergy (OoS), at which the myofibrils in the left heart chamber starts contracting isometrically and hence develop force rapidly which leads to an exponential pressure increase before ejection. OoS occurs within the pre-ejection interval, after the earliest mechanical activation and before aortic valve opening. OoS is therefore otherwise independent of the electromechanical coupling interval and the pre-ejection interval/isovolumic contraction period. By identifying how this point in time changes with therapy, it is possible to determine not only if a given therapy method would be effective in improving the prognosis of a patient, but also what would be the most effective therapy. A simple visual representation of an advancement of a measure that directly relates to the point of OoS, and how it varies with various kinds of pacing that may then be used to determine that BIVP would be the most effective treatment in this example may be seen in FIG. 41.

Whilst several methods are identified herein that allow for the point of OoS (or a similar point that directly relates to OoS) to be identified, such an identification requires unconventional data analysis steps that have been outlined herein to allow for detection, from which reliable conclusions can be reached. For example, the methods and systems described herein will only produce meaningful results under conditions were knowledge of the heart rate is known, knowledge of conduction through the AV node, knowledge of the time from stimulating the atrium either intrinsic or artificial to activation of the heart (whether intrinsic or artificial), or knowledge of the exact surface ECG configuration, so that if stimulation (intrinsic or artificial) is performed it can be recognized in the surface ECG or by VCG or electrical activation patterns of the heart.

Stimulation needs to be performed to avoid other activation than that from stimulation, calculated based on the knowledge above. For example, when stimulation from one electrode is performed, it should be tested that the stimulated heartbeat is that from stimulation and not that from intrinsic, as a combination of stimulations may lead to an inaccurate measurement of the time OoS,

When a new electrode is stimulated, again it should be checked that the stimulated heartbeat is that from the stimulus only, and not from intrinsic, premature, preexcitation or other stimulation. Similar considerations are to be taken into account before stimulating two electrodes or more in combination. Measurements of OoS can only be made on beats where the measured responses result from the stimulated electrodes, and where the measured responses change when stimulation is removed.

When configurations (i.e. non-synergistic pacing from is performed and pacing of one or more electrodes) occur later than the earliest recognizable intrinsic activation of the heart, then this earliest activation should be used for reference rather than that resulting from the artificial stimulus.

By taking the above factors into account, not only when pacing the heart but also when analysing the sensed and measured data, it is possible to obtain knowledge of a potential electrode position and configuration that can be used to program an implantable pacemaker to provide synergistic stimulation of the heart.

Electrode Positioning Using Cardiac Parallelity

By measuring the degree of cardiac parallelity (i.e. the degree of parallel activation of the myocardium), it is possible to characterize cardiac synchronicity as well as identify anatomical pacing zones that result in more parallel activation of the myocardium to reduce cardiac dyssynchrony (resynchronization). Such a measure may be utilized to guide and optimize CRT.

Firstly, in order to measure the degree of cardiac parallelity, a recruitment curve is generated, showing the area of the heart that is recruited following pacing from an electrode against time. From such a graph, the degree of parallelity may be determined.

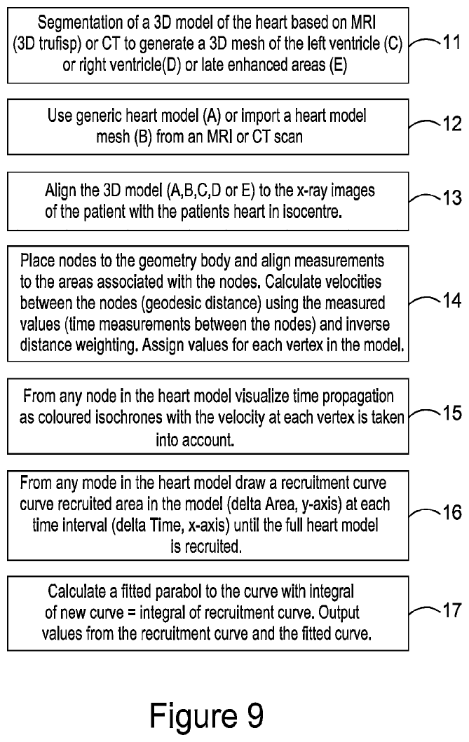

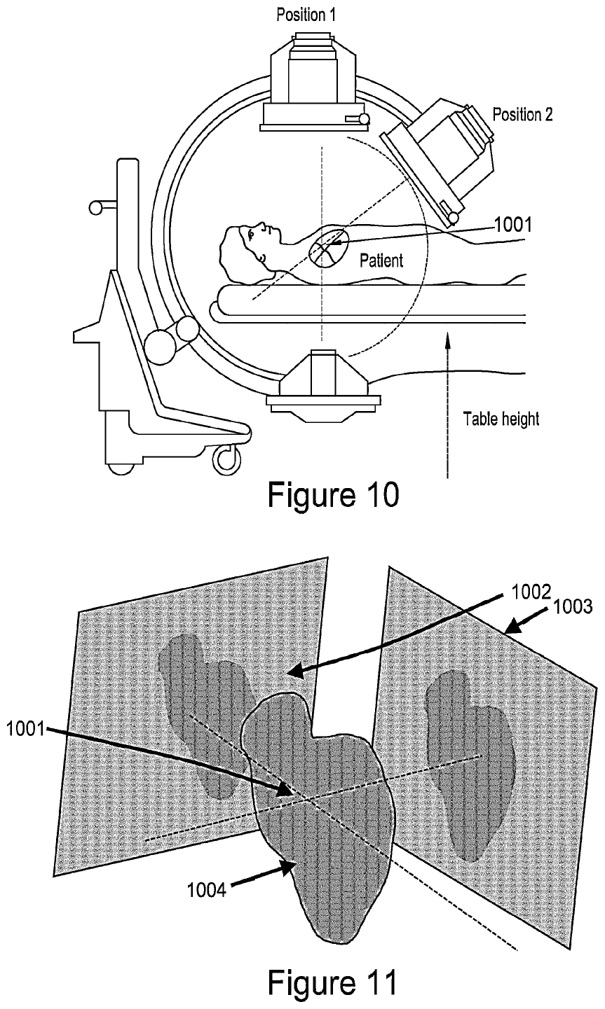

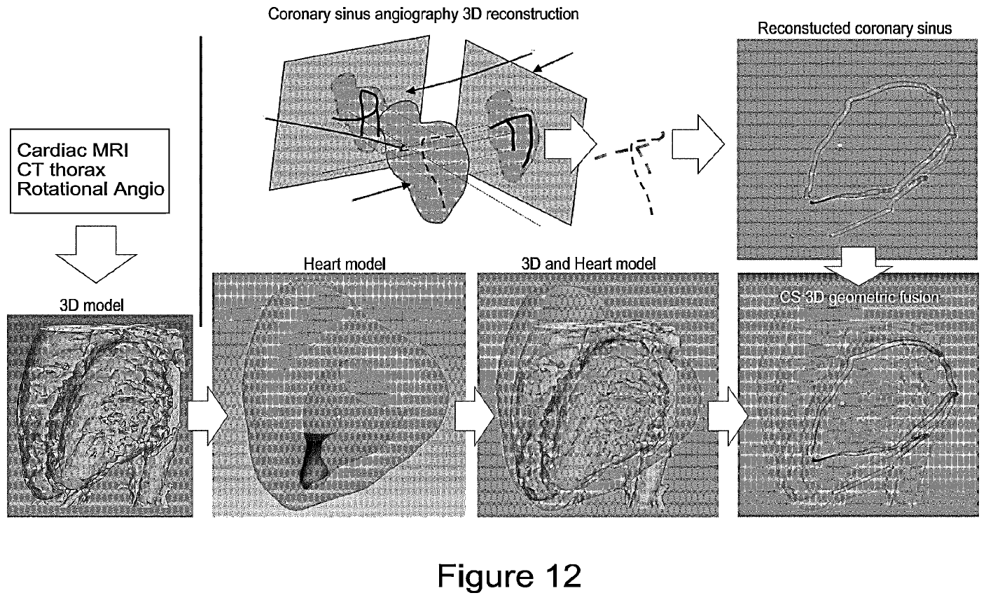

With reference to the method 10 of FIG. 9, a 3D model of the heart may be generated using medical images such as an MRI scan or a CT scan to generate a 3D mesh of the left ventricle, right ventricle and of late enhanced areas in step 11. Alternatively, the method may use a generic heart model, or a heart model mesh imported from a segmented CT/MRI scan, as in step 12. The 3D model of either steps 11 or 12 is then aligned to x-ray images of the patient, with the patient's heart at the isocenter 1001. One such method of aligning the 3D model with the heart of the patient may be seen in FIG. 10. At least two x-ray images 1001, 1002, as seen in FIG. 11, are taken at a known angle relative to each other, and are aligned relative to the fluoroscopy panels and to the isocenter 1001 in order to produce a 3D heart geometry 1004. Using the at least two x-ray images, the coronary sinus vein in 3D may be reconstructed as seen in FIG. 12. Using fluoroscopy panels and their known angles relative to each other with the patient's heart at the isocenter 1001, the coronary sinus vein may be reconstructed and overlaid over the 3D heart model of either steps 11 or 12.

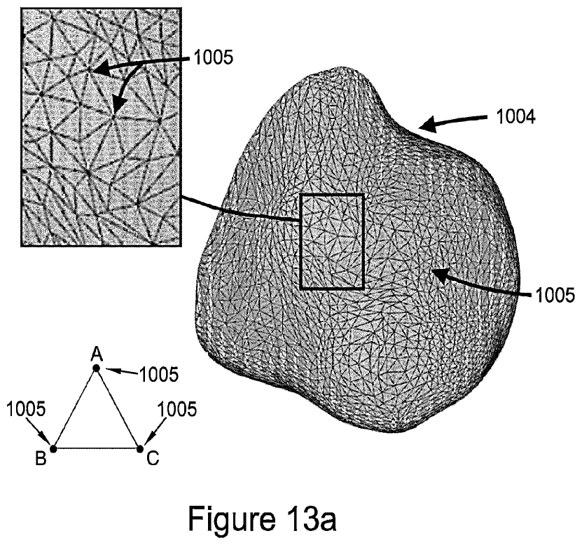

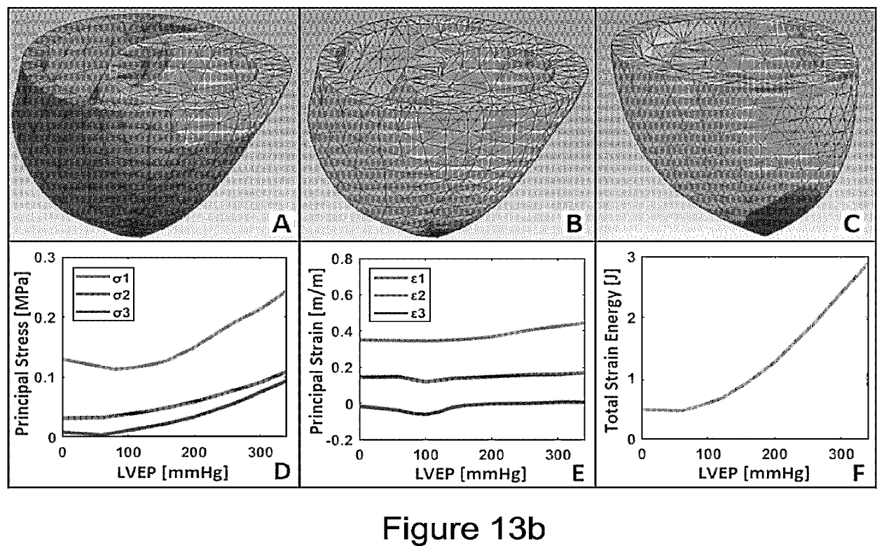

As can be seen in FIGS. 13a and 13b, the heart model 1004 (either a generic heart model or a specific heart model based on an MRI scan) may be converted into a geometric model consisting of multiple nodes (vertex) 1005 connected in a triangular network (vertices), representing a surface (FIG. 13a) or a volume (FIG. 13b). Electrodes 1006 may then be implanted into the heart, and during or after implantation additional nodes are marked on the geometry of the heart reflecting the positions of the implanted electrode. Between the nodes, intervals are input that reflect electrical intervals as measured by the electrodes in the patient when one of the electrodes are stimulated (paced). As will be understood by the skilled person, it is envisaged that the electrodes have already been implanted into the patient, and a heart model may then be updated to include nodes located at the points that the electrodes are located. A mathematical interpolation (e.g. inverse distance weighting) can be performed to assign values to the nodes between nodes with already measured values. In this way all nodes in the model will have values based on the measured values and the calculated ones to reflect electrical activation in the model. Calculation of electrical activation can be updated when new measurements are performed between electrodes, or modified with identification of areas of scar and/or fibrosis and/or other barriers to electrical propagation. The calculated values of all nodes is performed in such a way that electrical activation between all nodes in the model are at least partly explained.

The resulting geometry then contains multiple nodes with electrical time intervals measured between them and assigned to them. As the geodesic distance between all nodes may be calculated and calibrated, the geodesic propagation velocity of the electrical activation may then be calculated. The propagation velocity is then input to all existing nodes in the heart geometry (step 14).

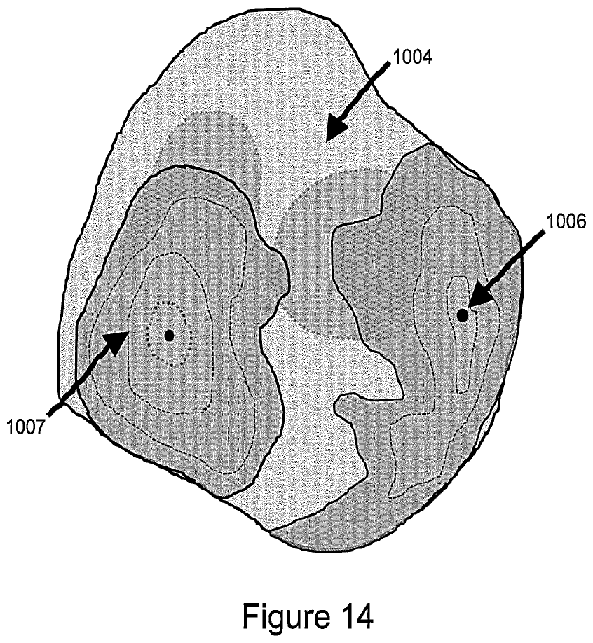

In step 15, the propagation from multiple nodes or electrodes 1006 may then be calculated, resulting in a visualization of time propagation of electrical activation throughout the heart as coloured isochrones 1007, taking velocity at each vertex of the heart model mesh into account as can be seen in FIG. 14.

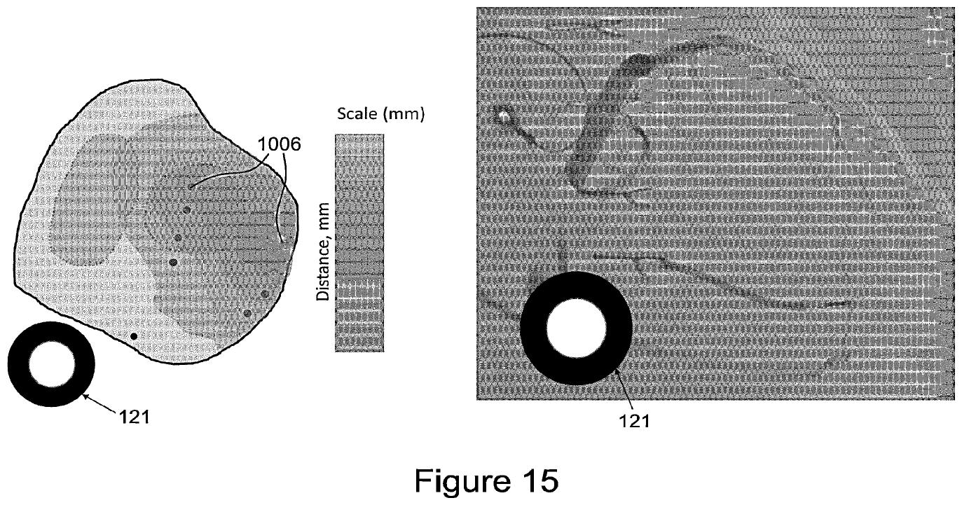

The geodesic distance between each node of the patient may be calculated. With reference to FIG. 15, an object 121 of a known size may be used on the fluoroscopy screen so as to calibrate the heart model for distance between vertices, which may then be represented and projected on the surface of the heart geometry as color zones and in a scale. In such a way, the heart geometry that is generated based on a generic heart model may be specifically tailored to each patient, with a known scale.

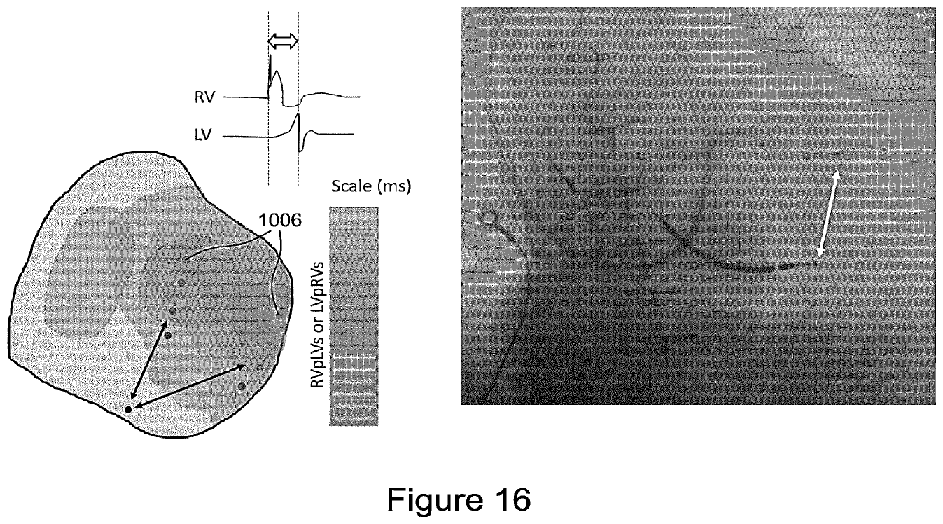

As may be seen in FIG. 16, by pacing at one node 1006 and sensing in the other nodes, it is possible to extrapolate measurements of recruited area of the heart and represent such measurements as color zones/isochrones. For example, as seen in FIG. 16, the right ventricle may be paced. The time delay from the pacing and then the sensing (RVpLVs) at another electrode can be used to assign time measurements to the known vertices. By utilizing the known geodesic distances between the vertices, it is possible to extrapolate said measurements to the other vertices of the heart geometry and thereby produce isochrones of the additional recruited area at a given time point. Therefore, these isochrones are based on measurements acquired from the specific heart of a patient from the implanted electrodes and are projected onto the model or patient specific reconstruction of coronary sinus vein. This allows for a patient specific heart geometry for visualization of numbers and allows further calculations to be taken into account using already known values of vertices and any number of vertices in between.

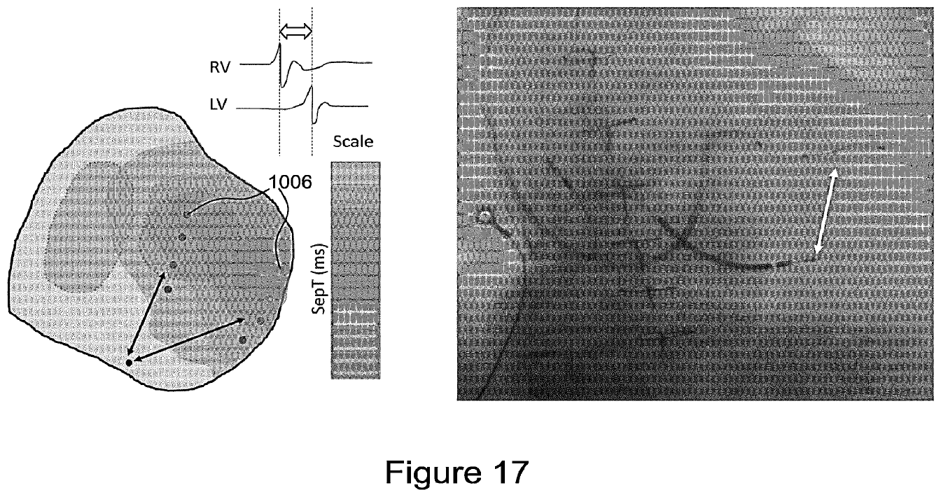

A similar process may be performed using separation time, as seen in FIG. 17. In this case, the heart is not actively paced, rather isochrones are generated on the heart geometry based on the separation time (SepT), i.e. when the electrodes 1006 are activated due to the natural pacing of the heart.

Using a combination of one or more of the measurements described above, it is possible to build additional compound measures and present them on a geometric model of the heart of the patient.

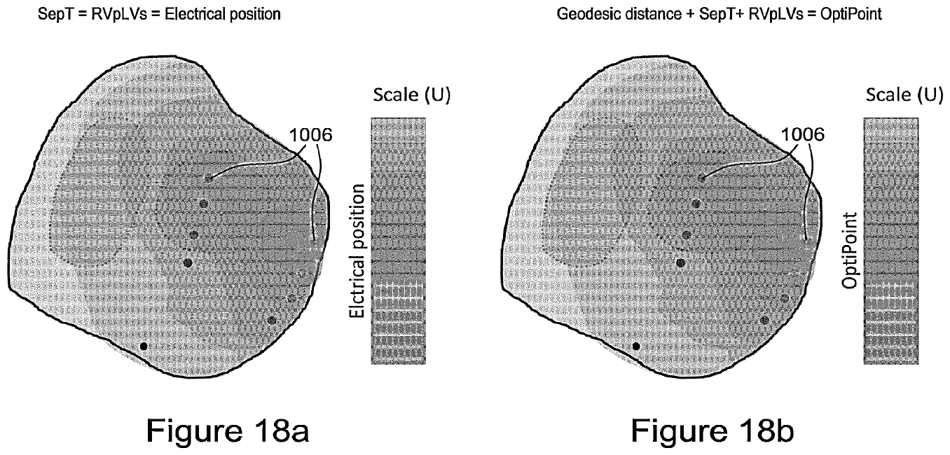

For example, as seen in FIG. 18a, a calculation based on SepT+RVpLVs may be calculated. Herein, such a measurement is termed “electrical position” and the calculation of this value provides different color representations of the heart model associated with certain regions of the heart (such as apical, anterior, lateral) for measurements obtained with the right ventricular electrode in the apex of the right ventricle.

By further adding geodesic distance, as in FIG. 18b, the optimal electrical and anatomical position may be considered. By such a measure, the result with the highest number on the scale representing a potential optimal (OptiPoint) position of an electrode. Such a position will represent the area most remote from present electrodes with the greatest effect. Such a placement of an electrode will achieve high parallelity when activated together with the right ventricular apical positioned electrode. Positions corresponding to the highest OptiPoint value are highlighted on a heart model, such as that of FIG. 18b, as being an area for potential electrode placement.

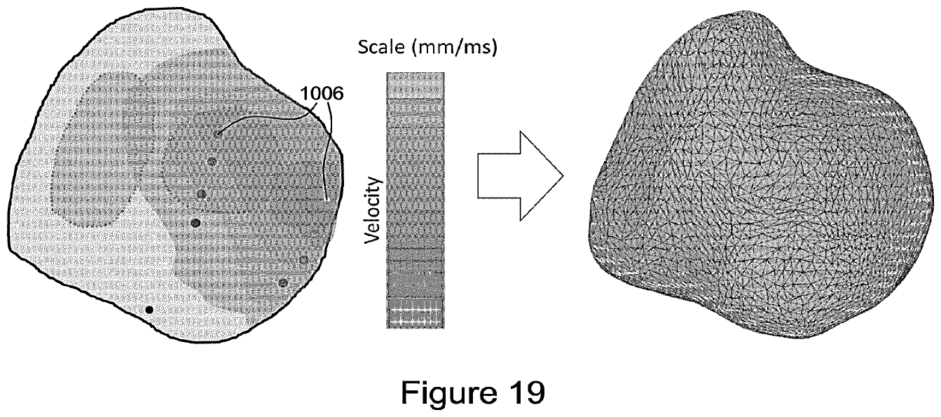

As seen in FIG. 19, measurements of time intervals from pacing one electrode to sensing at another electrode, in combination with the geodesic distance between the electrodes allows for calculation of geodesic velocity. Such a geodesic velocity may provide input to an inverse weighted interpolation algorithm/calculation to provide velocity values to all vertices in the model. In this way, velocity values can be extrapolated to all remaining vertices with no nodes attached, which can then be indicative of characteristics of the heart tissue. For example, each vertex may be assigned a value for its specific velocity that has been calculated using an inverse distance weighted interpolation taking into account the geodesic distance between the target and source nodes, as well as the number of neighbouring vertices. These values can then be used to extrapolate velocity values to vertices with no nodes attached.

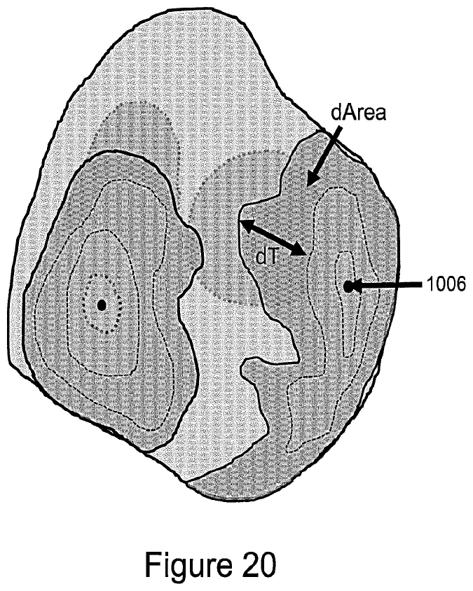

When the velocity at each vertex has been interpolated as outlined above, the propagation of electrical activation from the nodes may be represented on a heart model, as seen in FIG. 20. This allows for the propagation of electrical activation to be visualized based on the tissue characteristics as isochrones on a color scale on the model of the heart. Such a time propagation may show a change in area over a change in time, and can be visualized from single, or multiple nodes 1006.

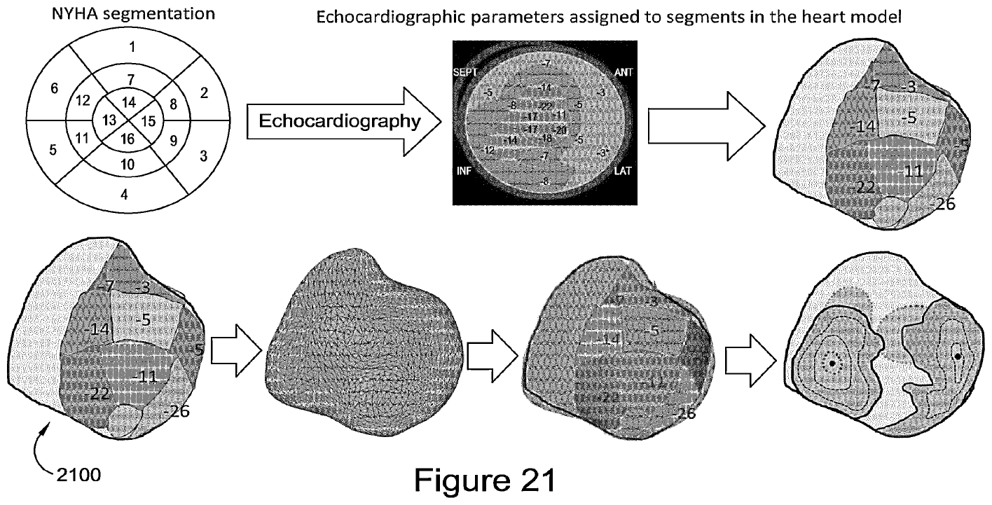

Further, echocardiographic data using segmentation may be transferred onto the heart model, and be used to modify and enhance the tissue characteristics of the heart model. For example, as shown in FIG. 21, using American Heart Association (AHA) left ventricular segmentation model or similar, echocardiographic parameters may be assigned to segments in the heart model and transferred to the vertices of the heart geometry. Such an assignment can be applied on to the existing vertices of the existing heart model and be used therefore to further classify all of the nodes of the geometry, as seen in the flow chart 2100.

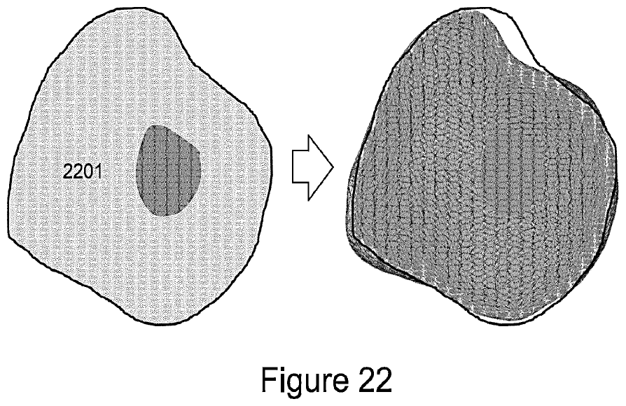

Similarly, scar tissue 2201 of the heart muscle, such as that which may be identified by a 3D MRI scan may be used to assign tissue characteristics of the heart geometry. This is further visualized in FIG. 22, wherein the area of scar is projected onto the heart geometry, and each vertex is assigned a value for velocity, enhancing the tissue characteristics. Such classifications may be utilized to modify a velocity model and assign new velocity values to the vertices that have been identified with additional tissue characteristics.

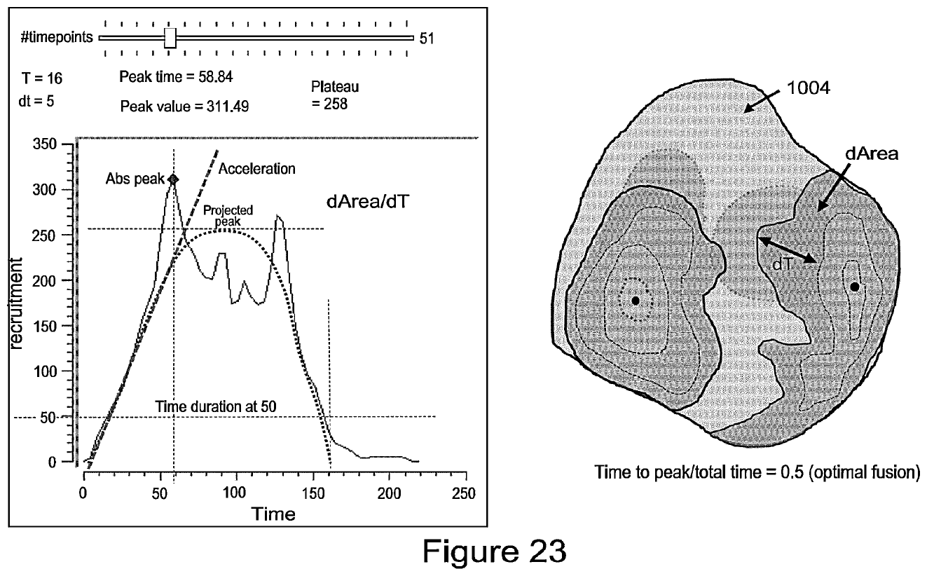

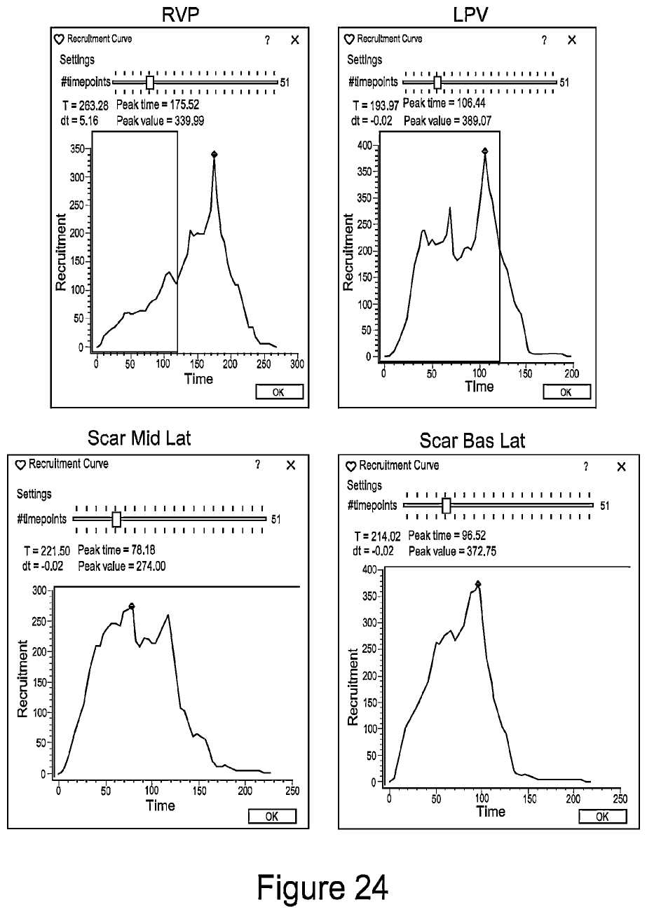

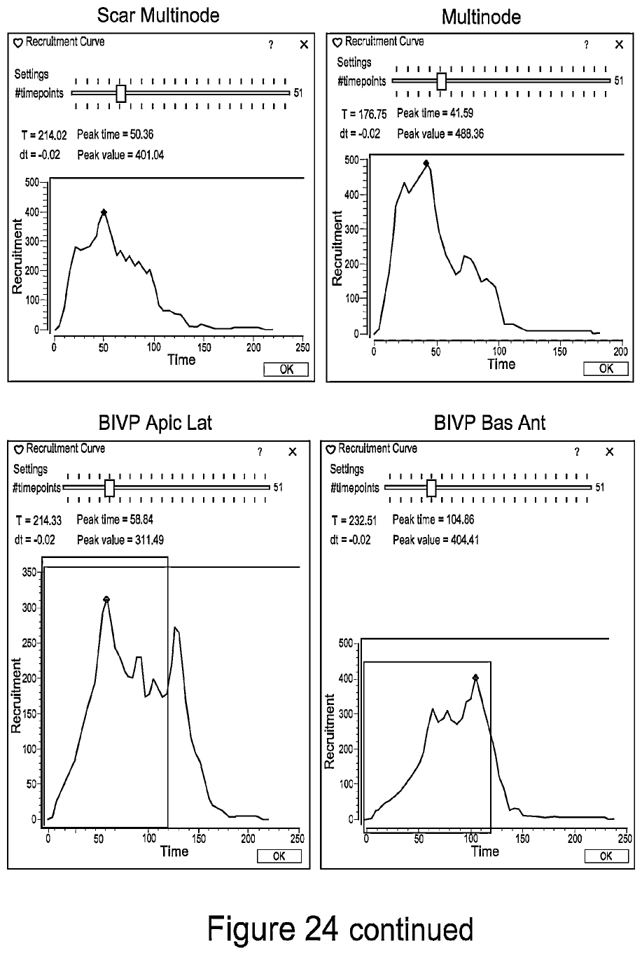

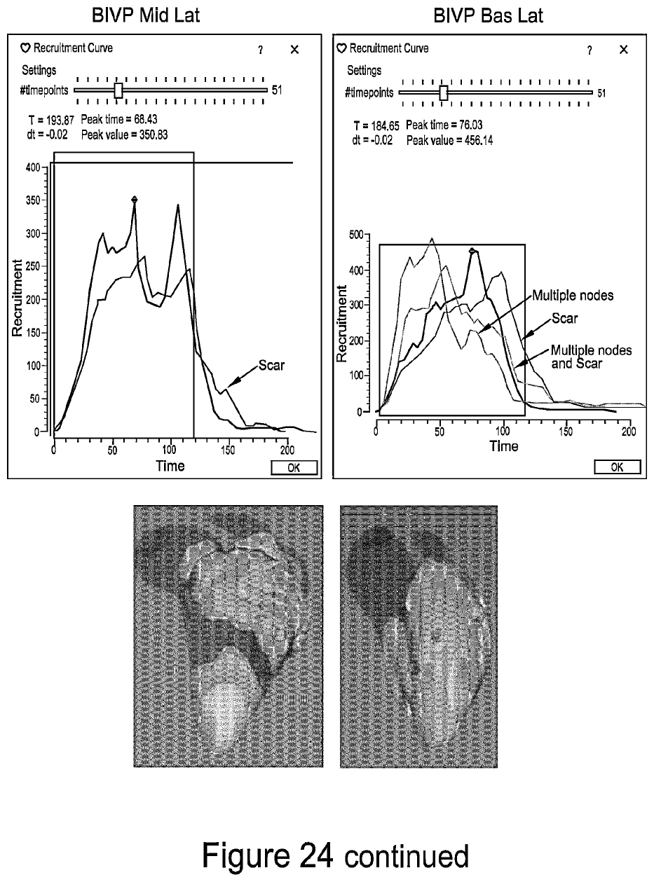

In step 16, the additional recruited area (of activated sarcomeres) at each point in time from the calculated velocity models can be calculated from multiple electrodes and the recruitment curve for said electrode(s) can be drawn based on the time propagation in the heart model when considering the added area at each time step until the full area, or a limited area, of the model is covered in isochrones, and their propagation from time=0 to time=x+1, as can be seen in FIGS. 23 and 24. In other words, the recruitment curve represents the recruited area or volume in the heart model with a measure of the change of area or volume of recruitment on the y-axis, and a scale of time on the x-axis. The recruitment curves can be characterised by multiple features, for example, the duration, slope, peak, mathematical expression, template matching.

Given the recruitment curve for a given node, a parabola may be fitted to the recruitment curve as can be seen in FIG. 23 and as described in step 17. The acceleration, peak and time to peak values of the propagation velocity can thereby be extracted from each recruitment curve, as well as the time to full recruitment (i.e. the time until the full heart model is recruited). More parallelity can be seen with a shorter time to peak propagation velocity, and thereby more propagation acceleration, as well as a larger peak value and a shorter time to full recruitment. Optimal curve characteristics can be provided, such that the peak recruitments should occur preferentially at 50% of the total recruitment time. The electrodes that create more parallelity (i.e. the greatest amount of total area of activation when the activation fronts meet) are chosen.

As can be seen in FIG. 24, the propagation curves may change with a change in electrode location and with the presence of scar. A number of recruitment curves are shown, and how each one varies is displayed for comparison. Based on such a comparison, the electrodes that result in the most ideal response may be chosen for pacing.

If the sensed activation pattern indicates too slow propagation through the tissue, the geodesic velocity is below a threshold, or the inability to provide sufficient parallel activation in the presence of scar tissue, the implantation of a CRT device should not take place, as such symptoms are not representative of dyssynchrony that may benefit from resynchronization therapy.

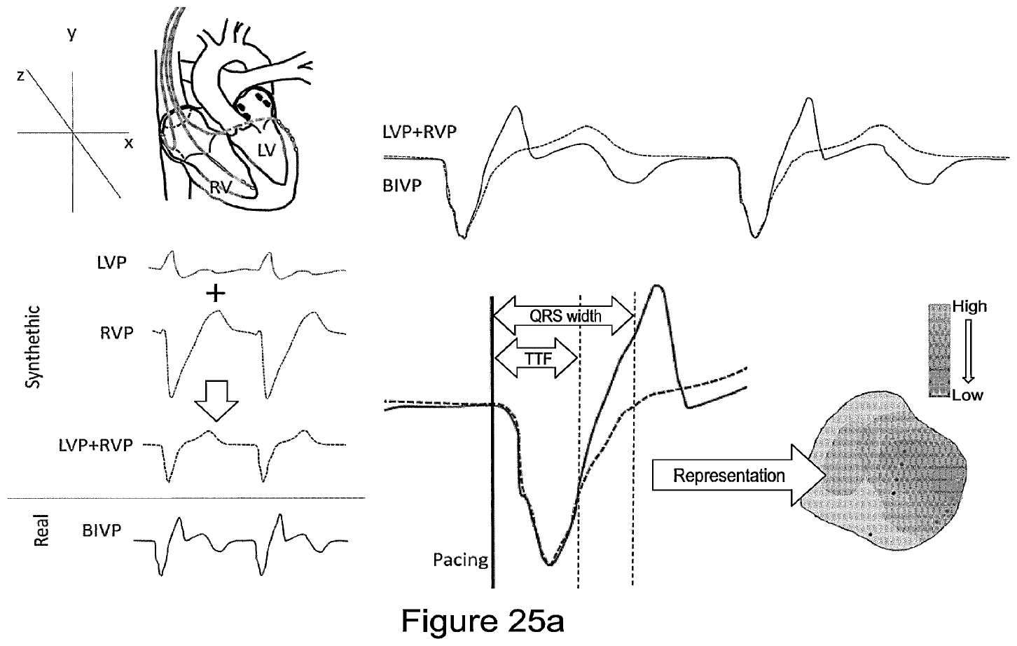

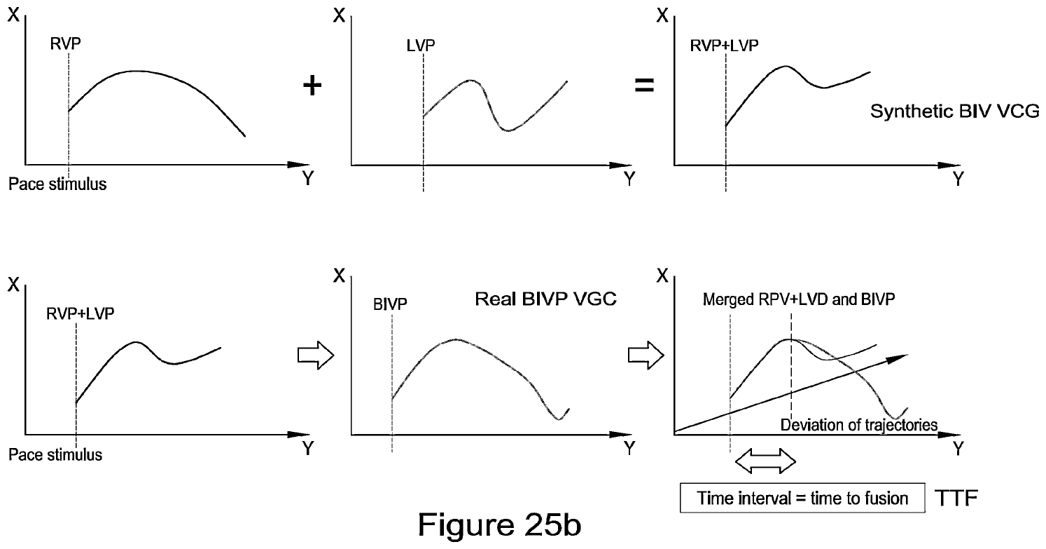

With pacing from each of the electrodes, a vectorcardiogram (VCG) recording the magnitude and the direction of the electrical forces that are generated during pacing of the heart is created. For each position that is tested, pacing is performed at each electrode, as well as for the two electrodes in combination, and a VCG is created for each situation. As seen in the example of FIG. 25b, a VCG RVp may be created for an electrode performing right ventricular pacing (RVp), and a VCG LVp may be created for an electrode performing left ventricular pacing (LVp). A synthetic VCG LVP+RVp may then be calculated from the sum of two of the created VCGs, and the real VCG is obtained when biventricular pacing is performed from the electrodes in combination, and collecting the resulting VCG BIVp.

The synthetic VCG LVP+RVp and the real VCG BIVp are then compared, as seen in FIG. 25a, and the point in time of deviation of the curve trajectories from each other is noted and the interval from onset of pacing to the point in time is calculated as a time to fusion time interval. Whilst the examples shown in in FIG. 25b are displayed in 2D, it will be appreciated by the skilled person that the comparison may occur in 3D in order to improve accuracy.

The time interval between the pacing stimulus and the point of deviation of the curve trajectories represents the time to fusion (i.e. the time until the electrical propagation in cardiac tissue from multiple sites meet). The longer period of time until the point of deviation indicates more parallel activation of the myocardium. Therefore, the time to the point of deviation between the synthetic and the real VCG should be as long as possible. The time to fusion may be calculated in isolation, or relative to QRS width to determine the degree of synchronicity (parallel activation).

A similar method may be performed with electrograms (EGMs) and electrocardiograms (ECGs) in one or multiple dimensions. If adding an electrode stimulus site does not shorten the time interval to deviation of the curve trajectories, or if the time to deviation increases; an additional benefit of adding the electrode is seen, such that the electrode can be added to the stimulation site and number of electrodes.

The method allows analyzing the additional effect of adding one electrode and compare this new state of pacing an additional electrode to the state of not pacing this electrode. If the new electrode does not decrease time to fusion, this indicates that the addition of this electrode allows capture and activation of tissue without promoting fusion at an earlier stage than without. Thus, more parallel activation occurs when time to fusion does not decrease with adding an electrode.

Whilst the recruitment curves described above suggest positions for the electrodes, the generated VCGs may be further used to validate them. In this regard, VCGs and recruitment curves are measures of electrical activation that should reflect each other. When these measures are concordant, it gives validity to the suggested electrode positions and validity to the model. To this point, once good positions are found for the location of the electrode based on the generated recruitment curves, this position is then validated based on VCG. As would be appreciated by the skilled person, these measures are not necessarily only used in combination, rather each of the recruitment curves or determining the point of deviation may both be used individually to determine suitable electrode positions. Both of these measures reflect parallelity, the degree of parallel activation of the myocardium, and therefore may be utilized alone to identify anatomical pacing zones that result in more parallel activation of the myocardium to reduce cardiac dyssynchrony (resynchronization). Such a measure may be utilized to guide and optimize CRT.

An inverse solution ECG may also be utilized in addition, or as an alternative to using implanted electrodes to measure the degree of electrical activation. By utilizing data obtained from surface electrodes applied to patients, it is possible to extrapolate a map of electrical activation onto the heart model using an inverse solution approach, given that the heart model has been positioned in an anatomically correct position as described above and the relative electrode position to the heart model is correct and known.

In such a case, activation of each node in the heart geometry is seen relative to the distance from the first activated area, and therefore calculation of velocity can be performed for the model. This velocity can then be used to calculate recruitment curves. When pacing from a single electrode, the activation can be calculated, similar to the calculation of activation from a different electrode. These measurements can form the basis of propagation velocity calculations and recruitment curves.

In such a case, body surface electrodes are used to determine parallelity (i.e. the degree of parallel activation of the myocardium) by collecting surface potentials. Such surface potentials may then be extrapolated onto the heart model that has been aligned so as to be collocated with the actual location of the patient's heart, as previously described. Thereby, an inverse solution ECG activation map of the heart may be produced, and the activation map may be manipulated as described above in order to determine propagation velocity, and thereby the presence of dyssynchrony.

In order to obtain such an inverse solution ECG, a system may be provided with surface electrodes to acquire multiple surface biopotentials (ECG). The system may be configured such as to provide an inverse solution, in order to calculate electrical propagation on a segmented model of the heart, which can include scar tissue including scar. By utilizing the geodesic distance (from the heart model which is aligned with the patient's heart) in combination with the electrical propagation together, the system may be configured to calculate propagation velocity in the heart model based on the inverse solution electrical wavefront activation of the heart in combination with the geodesic distance. Once geodesic velocity is assigned to each vertex in the heart model, time propagation and parallelity can be measured from any and multiple sites in the model.

Further, the surface potentials may be incorporated in the cardiac model as a characteristic utilized to calculate propagation velocity from single or multiple points on the heart model. This, as described above with respect to measurements directly from electrodes implanted into the heart, allows for the generation of multiple propagation velocity curves in order to calculate the differences multiple different points. Using such a comparison between the multiple propagation velocity curves, it is possible to choose the ones having better acceleration, peak velocity or propagation time as an indication of the preferred location for placement of electrodes.

Example Method

The systems and methods described herein may be used both before and during treatment of patients with presumably dyssynchronous heart failure, with a resynchronization pacemaker (CRT) in order 1) identify the presence of an underlying substrate that identifies patients that are likely to respond positively (manifest resynchronization potential present) to, 2) identify optimal locations for placement of pacing leads/electrodes, and 3) validate placement of optimal electrodes and resynchronisation of the heart.

Patients are currently referred for implantation of a CRT pacemaker based on international guidelines that describe indication criteria. These criteria are based on inclusion criteria in larger clinical trials and, amongst other things, consists of symptoms of heart failure, reduced ejection fraction (heart function) and a widened QRS complex (preferably left bundle branch block) beyond 120-150 ms. However, currently only 50-70% of patients with one or more indications for treatment with a CRT actually respond to treatment. Reasons for these non-responders are multiple, but lead position, the underlying substrate (dyssynchrony), scar and fibrosis and electrode positions are the most prominent reasons. By improving the detection of the underlying substrate that indicates dyssynchronous heart failure, it is possible improve the selection of responders (in a diagnostic capacity) for optimization of treatment (allowing therapy to be personalized to the patient).

Firstly, it is desirable to detect and define the underlying substrate (resynchronization potential) that defines whether a patient will respond to CRT, and whether the substrate is present or not in patients with standard inclusion criteria. When the substrate is present, one should proceed implantation of a CRT pacemaker, but when the substrate is not present one should follow other guidelines that apply.

When underlying substrate is present, or even if the underlying substrate has not yet been identified, an optimal position for the leads may be found, based on measures of parallelity, which takes scar and fibrosis into account. The measurement of parallelity is performed with guidewires or leads with electrodes inside the heart (for example, in veins or chambers of the heart). Optimal positions are for the placement of the electrodes is then suggested.

When the leads are in optimal position, according to the determined optimal position taking into account the measured parallelity from each node, it is then possible to confirm the response (by either direct or indirect measurements of onset of myocardial synergy), or alternatively reject the position.

If the desired response is confirmed, then a CRT pacemaker should be implanted. If the response is not confirmed, the mapping and measurements of parallelity should be refined before final confirmation. If response is not able to be confirmed, the implantation should be abandoned and known guidelines should be followed for alternative implantations.

It is envisaged that all of the methods and systems described herein may be used together, or equally may be used separately. In this regard, it is possible to detect the presence dyssynchrony and resynchronization potential, and confirm resynchronization without selecting the optimal lead position, and equally, it is possible to select optimal lead position without confirming underlying substrate and resynchronization.

Therefore, a system may be provided that includes connection to electrodes that allow visualization of signals from the patient and measurements time intervals. Alternatively or additionally, a system may also be provided that includes sensors and electrodes and allows visualization of a heart model and calculations based on the heart model's geometry. Both of the above systems can be combined in the operating room.

An implementation of the above systems and methods will be further described herein by way of an example implementation during surgery.

A patient is firstly taken in to the operating room and sensors and electrodes are fixed on the patient's body surface.

In order to determine the delay to onset of myocardial synergy (OoS), one or more additional sensors may be utilized. For example, one or more of a pressure sensor, piezo-resistive sensor, fibreoptic sensors, an accelerometer, an ultrasound and a microphone may be utilized. Measurements from the additional sensors may be taken in real-time and be processed on location. If the delay to onset of myocardial synergy is short relative to the QRS complex or short in absolute values (for example either shorter than 120 ms or less than 80% of the QRS duration), then the implantation of a CRT device should not occur. When the delay to onset of myocardial synergy is measured to be long compared to the QRS complex or long in absolute values (for example either longer than 120 ms or longer than 80% of the QRS duration), then implantation of the CRT device should occur.

Body surface electrodes are used to determine parallelity (the degree of parallel activation of the myocardium) by collecting surface potentials for an inverse solution ECG activation map of the heart as described above to determine propagation velocity, and thereby the presence of dyssynchrony. Additionally or alternatively, electrodes implanted within the patient's heart may also be used to produce electrical activation maps, and thereby determine the presence of dyssynchrony. If the sensed activation pattern indicates too slow propagation through the tissue, or the inability to provide sufficient parallel activation in the presence of scar tissue, the implantation of a CRT device should not take place.

The patient is then prepared for surgery and sterile draped. Surgery is started as usual and leads are placed in the patient's heart through a skin incision below the left clavicle and puncture of the subclavian vein. The leads are then moved into position in the right atrium and right ventricle.

Dyssynchrony may then be introduced by pacing the right ventricle, and can be confirmed when measuring the delay of myocardial synergy as discussed above. A sensor may be placed in the left heart chamber, or in the right heart chamber, in order to determine the delay of onset of myocardial synergy. In this way, the same calculation may be performed as previously utilized in order to calculate the delay to onset of myocardial synergy.

Once the leads are in position, the coronary sinus is cannulated and an angiography in two planes are performed to visualize the coronary veins.

Once the coronary vein is visualized, cannulation can be performed with either a thin guide wire with an electrode at the tip, or any catheter with one or multiple electrodes for mapping purposes. Measurements of time intervals are then used to characterize one or more of the intrinsic activation, tissue properties and vein properties. The coronary anatomy is then reconstructed in software, and measurements are assigned to positions in the heart model relative to the reconstructed coronary sinus vein.

This data may then be used, in a method performed outside of the body, to calculate parallelity in order to highlight the electrode positions with the highest value of parallelity. Based on these measurements, the surgeon is advised to position the left ventricular (LV) lead with electrodes in a desired position/vein. Similar advice can be given also to reposition the right ventricular (RV) lead. Based on the acquired measurements and the processing thereof, advice can also be provided to include other and/or further electrodes to achieve a higher degree of parallelity. Other electrodes refer to other electrode positions than those available (endocardial, surgical access), and further electrodes refers to the use of multiple electrodes (more than two).

As a result of the above, the coronary vein branches are now seen in two planes and a suitable vein is selected for placement of a left ventricular lead.

When the LV electrodes are in position, the sensors may be used to determine the delay to onset of myocardial synergy, when pacing both the RV and the LV. Different electrodes may be analyzed by repositioning the LV lead at different positions. Measurements of the delay to myocardial synergy may occur using one or more of a pressure sensor, piezo-resistive sensor, fibreoptic sensor, an accelerometer, an ultrasound or by measured bioimpedance (when connected to the RV and LV leads). If the delay to myocardial synergy is not shortened, at least to less than for example 100% of the intrinsic measured value or when the bioimpedance measurements indicate by paradoxical movements that resynchronization is not taking place, the proposed lead positions should be abandoned. The intrinsic value measured from the QRS onset does not include the time from the onset of pacing to ventricular capture, and hence is by definition shorter than that measured from the stimulus. 110% would therefore approximate the time interval measured with intrinsic activation. In this way, the intrinsic delay to onset of synergy measured from the QRS complex can be calibrated by adding, for example, 15 ms to the value reflecting the time from pacing spike onset to electrical tissue capture that occur when artificially pacing.

When pacing the RV, the LV or both, a VCG can be reconstructed and the time to fusion can be calculated. The time to fusion may further be used in order to confirm the already measured parallelity. Surface electrodes can be used for inverse modelling to measure time to fusion. If the measured time to fusion, and the measured parallelity does not concur, the causes of such a discrepancy should be further reviewed.

It is possible that LV leads with multiple electrodes can be used on the discretion of the physician. The use of multiple electrodes can be used in measuring parallelity, and when found to increase parallelity, such an increase in parallelity can be confirmed using time to fusion, and by measuring the delay to onset of myocardial synergy.

Once the lead is in desired position, wherein the delay to onset of myocardial synergy is less than (for example) 110% of initial intrinsic value and less than (for example) 100% of the biventricularly paced QRS complex and, the CRT may be implanted and the device generator connected and implanted in a subcutaneous pocket. If the lead is found not to capture the myocardium or if the location is determined suboptimal based on scientific empiric data or measured intervals (QLV), the lead is repositioned and retested before the device generator is connected. The skin incision is then sutured and closed.

The systems described above may be embodied in an overall system that contains a signal amplifier or analogue digital converter (ECG, electrograms and sensor signals), a digital converter (sensor signals), processor (computer), software, connector to x-ray (either by direct communication with a dicom server or PACS server, or indirect with a framegrabber and an anglesensor). It is possible to use the system with different sensors at user discretion. Further, the system may also be used to solve other problems as well. For example, the systems may be utilized for identification of His region and placement of a pacing lead in the His bundle, with additional measurement of the delay to onset of myocardial synergy.

Example System

Also provided is a catheter than can be used in the methods described above. In this way, a catheter is provided with a system that can be used to detect dyssynergy caused by dyssynchrony, as well as to help select the right patient for therapy. The catheter may comprise a cardiac catheter with a lumen for guidewire and saline flush. The catheter comprises one or more sensors. For example, the catheter may comprise vibrations, pressure, acceleration, and electrodes for sensing electrical local and global cardiac signals. The catheter can be placed in the left or right heart chamber through venous or arterial access, and/or in the coronary vein. Electrodes can be used for sensing electrical signals in a bipolar or unipolar fashion (to a reference electrode on the catheter, or any other electrode connected to the patient body), and the electrodes can be used for pacing the heart at various positions. The catheter connects to a system for processing of the data, either through cables or wirelessly. A guidewire can be passed through the lumen of the catheter to increase the diameter of the distal curve, and a guidewire can be passed through the end of the lumen to get in contact with the cardiac tissue and be used as a sensing and pacing electrode.

When the catheter is passed into the heart chamber, it is possible to use the electrograms provided from the sensors of the catheter to measure the electrical delay from one electrode to the other (or to an electrode that is external to the catheter), and as such determine the electrical activation time. Additionally, using the catheter, it is possible to measure other factors such as vibrations, pressure and acceleration, and then filter the signals to receive measures that can be used to determine the onset of synergy in the heart. Therefore, the catheter can be used to obtain measurements that can be further used to measure the degree of resynchronization and the resynchronization potential. Equally, the catheter maybe provided as part of a system that, for a given set of electrode positions, can measure all data required to calculate the time to onset of synergy. Therefore, system comprising the catheter may be used to quickly and easily determine the resynchronisation potential of a patient.

Such a catheter may provide several uses. As considered above, the catheter may be used to obtain all measurements to be used to detect the onset of synergy following pacing, and determining the resynchronisation potential of a patient. For example, such a method for determining the onset of synergy is defined above, or in GB1906064.9. The catheter may find use in taking measurements to determine the degree of parallel activation. For example, such a method for determining the degree of parallel activation is described above, or in GB1906055.7. Equally, the catheter may be utilised to take measurements to determine the time to fusion in a heart. For example, such a method for determining the time to fusion in a heart is described above, or in GB1906054.0. The catheter may be provided additionally with a data processing module that can additionally process the data received from the catheter to provide a measure of any of the above values, without need for further post-processing of the data.

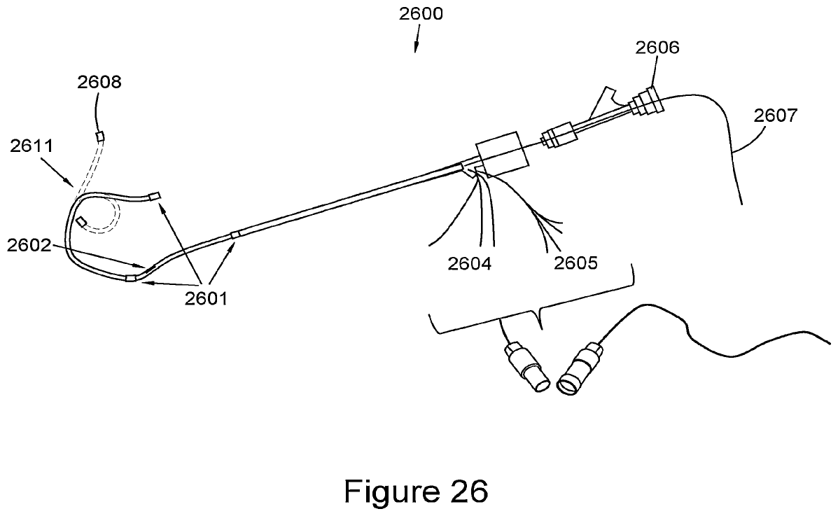

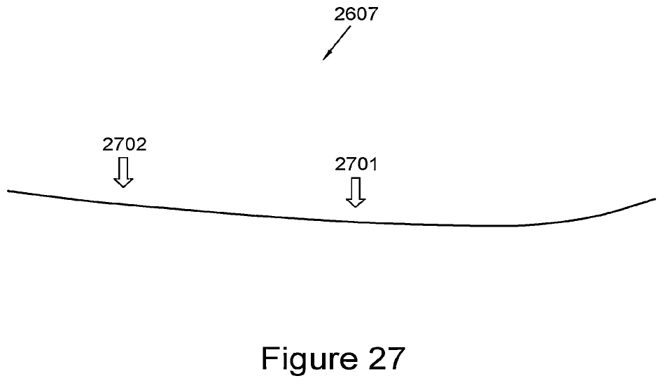

Such a catheter 2600 may be seen in FIG. 26. The catheter comprises one or more electrodes 2601, one or more sensors 2602, a shaft 2603, communication means 2604 and 2605, a hemostatic vent 2606, and a guidewire 2607. The catheter extends to a distal end 2608.

The sensors may be any desired sensor. For example, where the catheter is for use in determining the delay to onset of myocardial synergy, it may be desired that the sensor is a pressure sensor such that it is possible to invasively measure the pressure within the heart, and thereby measure the change of pressure within the left ventricle. Additionally or alternatively, the sensor may comprise a piezoelectric, fiberoptic and/or an, accelerometer sensor. The sensor may detect and transmit events such as cardiac contraction, onset of synergy, valve events, and pressure to a receiver connected to a processor.

The distal end 2608 of the catheter 2600 is a floppy pigtail, such that the electrodes 2601 positioned at the curved distal end may be moved by advancing the relatively stiff guidewire 2607 along the shaft of the catheter. By advancing the guidewire through the catheter 2600, the diameter of the curve provided at the distal end 2608 of the catheter 2600 is increased. This allows for the distal end 2608 of the catheter 2600 to be moved, and thereby allows for movement of the electrodes 2601. Such variable positions are shown in broken lines 2611 in FIG. 26. Additionally, the distal end 2608 of the catheter 2600 may be provided with a soft tip for atraumatic contact with the lateral wall endocardium.

Communication means 2604 may transmit data received from the electrodes 2601, and communication means 2605 may transmit data from the sensor(s) 2602. As shown, these may be provided as physical wires to plug into an external data processing module. Alternatively, they could provide wireless transmission, to transmit the data without a physical connection. The shaft of the catheter 2600 may be of any suitable diameter. For example, the shaft may be a 5 Fr shaft. A saline flush may additionally be provided through hemostatic vent 2606.

A more detailed view of the guidewire 2607 may be seen in FIG. 27. A stiffer body 2701 is provided at the proximal end of the guidewire 2607, and then a flexible tip 2702 is provided at the distal end. Such an arrangement allows for finer adjustment of the position of the catheter, and the electrodes and sensors that are positioned thereon.

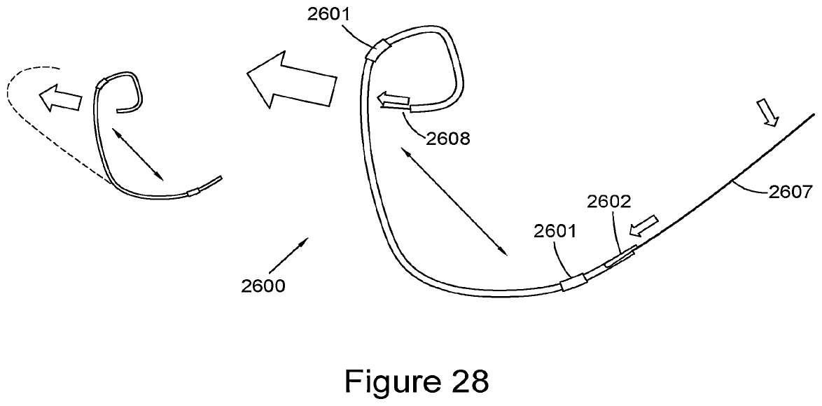

FIG. 28 shows how the guidewire 2607 may be used to manoeuvre the catheter 2600, and more specifically, the electrodes and sensors disposed thereon. As shown, the guidewire 2607 is introduced through the proximal end of catheter 2600. The guidewire extends through the catheter 2600 towards the distal end 2608. As can be seen, the catheter 2600 is a floppy pigtail shape such that when the relatively stiffer guidewire 2607 is advanced through the catheter 2600, the diameter of the curve provided by the catheter 2600 is increased, as seen in FIG. 28. The stiffer body 2701 near the proximal end of the guidewire 2607 provides a more pronounced enlargement of the curve of the catheter than the flexible tip 2702. This provides for more accurate control of the location of the electrodes 2601 (and other sensors 2602) on the catheter 2600.

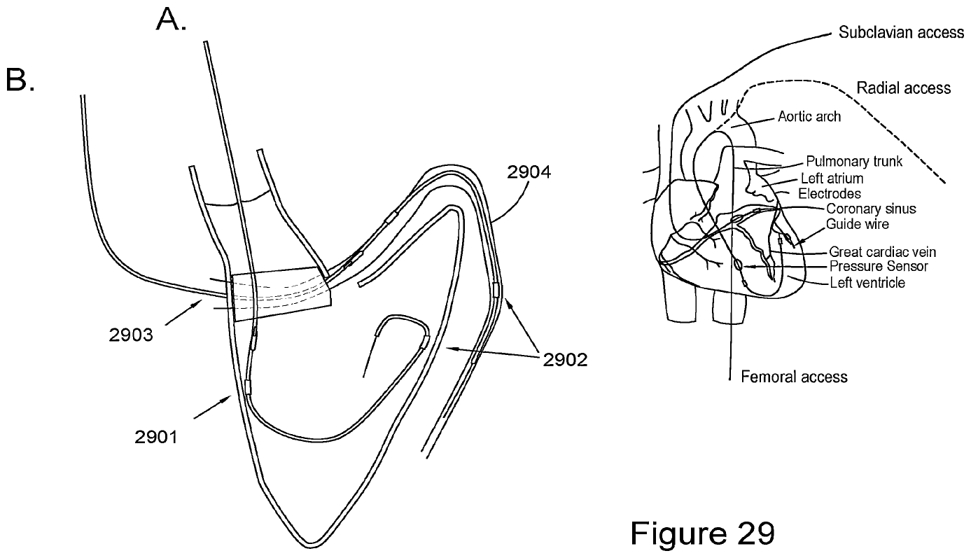

Various different locations within the heart in which the catheter 2600 may be placed are illustrated in FIG. 29. For example, the catheter may be provided through location A, providing arterial access into the heart chamber, or through location B, providing venous access to the heart chamber. Though location A, the catheter (and embedded sensors and electrodes) pass through the septum 2901 toward the contralateral wall 2902, such that electrodes may be placed in the septum and the contralateral wall. Through location B, the catheter may pass through the coronary sinus ostium 2903 and the coronary vein 2904, such that the catheter (and electrode(s)) is passed through the venous system into the coronary vein. Alternatively, the catheter may be provided through subclavian access, radial access or femoral access. The catheter is configured to be positioned in the left heart chamber, with the electrodes opposing each other at the septum and contralateral wall, with and the sensor provided within the chamber. The electrodes are to be provided in contact with the tissue.

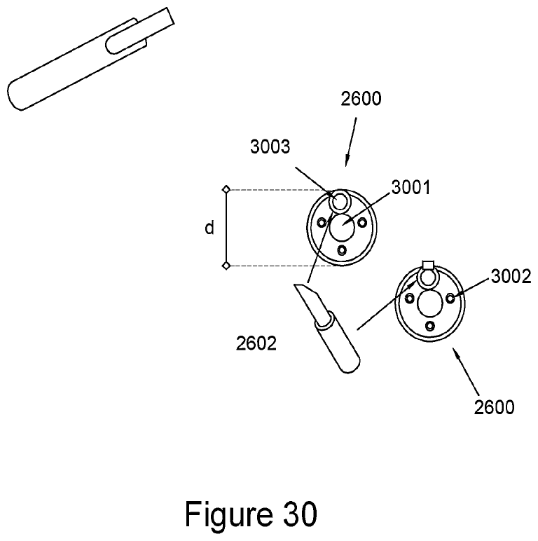

FIG. 30 shows two cross sections of the catheter 2600. As stated above, catheter 2600 may be provided at any suitable diameter d, such as 5 Fr. The catheter 2600 is provided with an interior lumen 3001 through which the guidewire may pass. Additionally, saline flush may be provided through the interior lumen 3001. Interior lumen again may be provided with any suitable diameter, such as 0.635 mm (0.025 inches). Catheter 2600 is additionally provided with a number of channels 3002 for electrode leads, and a number of channels 3003 for sensor leads, connected to embedded sensor 2602.

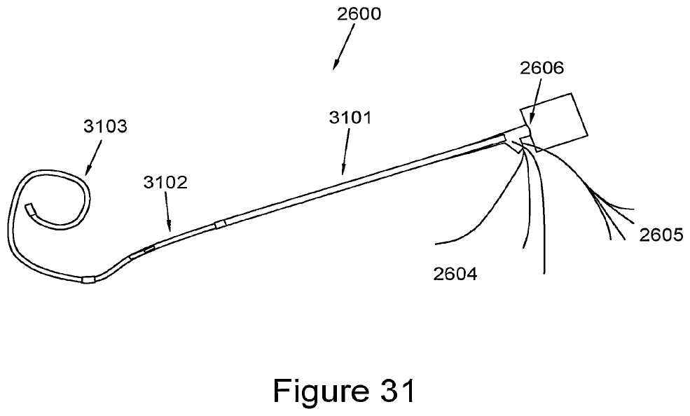

A more detailed view of the structure of the catheter 2600 is seen in FIG. 31. As described above, saline flush may be provided through hemostatic vent 2606. The catheter 2600 is provided with a stiff proximal end 3101, a middle part 3102 which is of an intermediate stiffness, and a flexible tip 3103 at the distal end of the catheter.

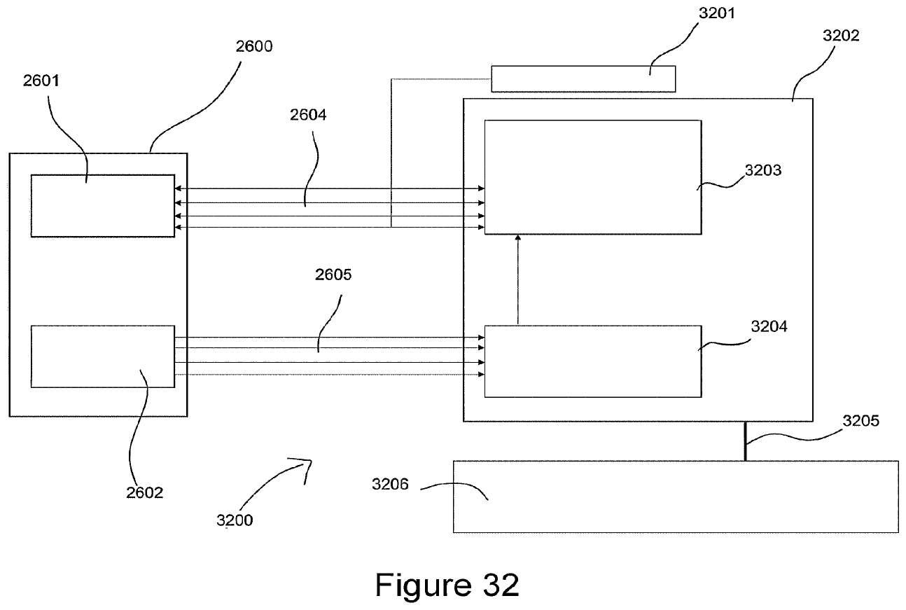

FIG. 32 shows a system 3200 for sensing and processing data comprising a catheter as described herein. The catheter 2600 is in signal communication with stimulator 3201, amplifier 3202 and processor 3206. As described above, catheter 2600 comprises electrodes 2601 and sensor(s) 2602. The electrodes are in signal communication with stimulator 3201 and analog converter 3203 of amplifier 3202 through communication means 2604. The sensor(s) 2602 are in signal communication with receiver and converter 3204, and additionally to analog converter 3203 of amplifier 3202. The amplifier 3202 then provides an output to a processor 3206. For example, the amplifier 3202 may be connected to the processor 3206 by means of a fiber optic cable 3205.

The processing module 3206 may be configured to take the data gathered by the catheter 2600 and further process the data so as to provide meaningful assessments as to the cardiac function of the patient. For example, the data processing module may be configured to calculate the delay to onset of synergy, the time to fusion or a measure of parallelity of the heart of the patient.

For example, the catheter may be provided with at least one piezo-electric sensor 2602 (and/or optical sensor 2602, and/or accelerometer 2602) that is configured to directly measure pressure within the heart. Utilising such information, the catheter 2600 and the processing module 3206 may be configured to automatically and reliably detect a point relating to the onset of synergy, which is distinct from and occurs at some point between the pre-ejection interval (PEI) and electromechanical delay (EMD).

For example, whilst this may be relating to a rapid pressure rise originating from the onset of synergy, the point of the onset of synergy may be better and more reliably represented by filtered pressure traces. Therefore, the system 3200, and more specifically the piezo-electric sensors 2602 of the catheter 2600 and processing module 3206 may be configured to detect the pressure change within the heart, and filter the pressure traces so as to give an accurate representation of the onset of synergy. This may be achieved by removing the first harmonics of the pressure wave by band-pass filtering at, for example, 2-40 Hz. This curve, as described above, has a linear upstroke that originates from the onset of synergy and that crosses zero at peak dP/dt. Filtering at, for example, a band-pass 2-40 Hz or 4-40 Hz removes the low, slow frequencies that are associated with dyssynergy and the onset of synergy may be seen as the onset of the pressure increase that leads to, or is directly prior to aortic valve opening or maximum pressure.

This change in rate of pressure increase is because of increasing and exponential cross-bridge formation while passive stretched segments tension increase, either because depolarization or because elasticity model reaches its near maximum. Rapid cross bridge formation with isometric or eccentric contraction leads to high-frequency components in the pressure curve frequency spectrum, which reflects onset of synergy. This phase of the cardiac cycle may be seen when filtering LVP with high pass filter above the 1st or 2nd harmonics. The filtered and characteristic waveform has a near linear increase, from onset of synergy to crossing 0, and continues with a linear increase up to aortic valve opening. The line of linear increase reflects the period with synergy, crossing zero at halfway in the phase, which corresponds to peak dP/dt as described above, and onset of synergy is reflected in where this line starts to rise above the floor of the filtered pressure curve or at its nadir. Additionally, the catheter 2600 and processing module 3206 may be configured to utilise high frequency components (above 40 Hz) of the pressure trace to identify the onset of synergy in the mid range filtered (4-40 Hz) signal as the high frequency components identifies the onset of pressure rise prior to zero-crossing.

One or more of these points in the pressure trace (the beginning of the linear increase in a band-pass filtered pressure trace, the crossing of zero in a band-pass filtered pressure trace, the onset of high frequency pressure components of the pressure trace), taking data that is filtered from the piezo-electric (or other optical) sensors 2602 of the catheter 2600 may be utilised by the data processing module 3206 to accurately and reliably represent the onset of synergy. Additionally or alternatively, the sensors 2602 may comprise accelerometers that gather accelerometer data within the heart, and from such data determine the onset of synergy, for example as described above and illustrated in FIG. 35. The raw acceleration data 301 may be band pass filtered resulting in data 3502, and from such data, a wavelet scalogram 3503 may be produced, which shows the frequency spectrum over time. The center frequency trace fc(t) 3504 is then calculated from the wavelet scalogram as seen in graph 3504. For each cycle of the heart, averaging each cycle and extracting the time of the peak fc(t), it is possible to determine the time-to-onset of synergy (Td) as seen in graph 3506. The time to onset of synergy may be measured from any suitable reference time, such as the QRS-onset, 3507.

As would be appreciated, any of the measures considered herein of detecting onset of synergy (or points relating directly thereto) may be combined to provide a more accurate measurement of the onset of synergy and/or how it varies with treatment. For example, a measure of the time of onset of synergy or a point related thereto before/after treatment calculated by filtering pressure data may be compared and contrasted with the point of onset of synergy calculated using raw acceleration data within the heart before/after treatment. In this way, a reduction in the time to onset of synergy (thereby indicating that reversible cardiac dyssynchrony is present) may be validated using more than one measure.

By utilising any of the above measures, the system may therefore, for each position of the catheter and therefore the electrode(s), automatically determine how time until the onset of synergy varies. In this way, the system can give immediate (or near immediate) feedback on the efficacy of various electrode placements in reversing dyssynchrony and dyssynergy.

In one example, as a representation of the time of onset of synergy, the zero crossing from a filtered signal or a template match from a filtered signal may be detected within a timeframe from a reference time. For example, the zero crossing within a timeframe of ±40 ms of QRSend (so as to ensure that the first zero crossing, being the zero crossing associated with the same heartbeat) is measured. Alternatively, the onset of synergy may be indicated by the timing of the nadir (i.e. the point of pressure increase from the pressure floor) together with high frequency components. As would be appreciated, both of these measures (and others) can represent the onset of synergy, being the point where all segments of the heart begin to actively or passively stiffen. This is practically manifested in the beginning of the rapid pressure rise within the heart.

Whilst the point of onset of synergy is manifested in the increase of pressure within the left ventricle due to the point where all segments of the heart begin to actively or passively stiffen, it will be appreciated by the skilled person that this point can also indirectly be measured in other positions. In this way, and in addition to positioning within the left heart chamber, the catheter may for example be positioned within the coronary veins or in the right heart chamber to provide similar measurements indicative of the onset of synergy, with appropriate filtering of the signal.

In sum, it may be said that the catheter measures pressures and/or vibrations, and can subsequently apply different filters for the assessment of the pressure/vibrations, together with the electrical signals detected by the catheter to determine if dyssynchrony is present or not. Whilst a reduction in the delay to onset of synergy (for example, calculated as described above) indicates that dyssynchrony is present, a prolongation of the interval with stimulation when compared to the baseline (i.e. a case with no stimulation) identifies an iatrogenic potential. Such a situation may be detrimental to the patient's health and should be avoided.

Sensor Calibration Effect on dP/Dt:

Advantageously, the sensors of the catheter may not require calibration for time events when using the derivative of pressure that relates to the measurement of onset of synergy.

In theory the offset and gain of the pressure signal should not affect the results of when dP/dt=0 or when dP/dt peaks. The offset will not affect when dP/dt=0 or when dP/dt peaks because the derivative of the offset will go to zero. While the gain will affect the value and slope of the pressure sensor signal, the gain will not affect the time the maximum/minimum of the pressure signal occurs (which is when dP/dt=0) or the time the maximum/minimum slope of the pressure signal occurs (which is when dP/dt peaks).

This effect is illustrated by the below simplified example demonstrating how neither the offset nor gain will affect a cyclical pressure signal.

For example, if the true pressure signal was characterized by the equation:

Pₜᵣᵤₑ=sin(60t)

And the catheter had an offset of 100 mmHg, with a gain of 5 times more than the actual signal. Then the pressure signal reading would be characterized by the equation:

P_{reading}=5 sin(60t)+100

Even given the differences in the true pressure signal and the reading pressure signal, the derivative of both equations with respect to time (t) would be:

$$\frac{d ( {P}_{true} )}{dt} = 60 cos ( 60 t )$$

$$\frac{d ( {P}_{reading} )}{dt} = 300 cos ( 60 t )$$

Whilst the amplitudes of the two dP/dt equations differ, the time when dP/dt=0 and when dP/dt peaks will be equivalent for both equations

$$( t = \frac{( 2 n + 1 ) π}{120} and t = \frac{n π}{60}$$

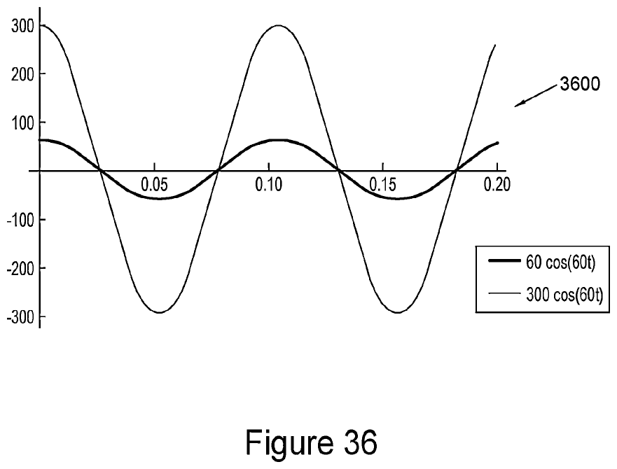

respectively, where n is the value of any integer). This is shown in FIG. 36, which shows a graph of the derivative of Pₜᵣᵤₑ and P_{reading} from the example given above. It can be seen from this example that dP/dt=0 and dP/dt peaks at the same times for both of Pₜᵣᵤₑ and P_{reading}.

It should be noted that signal changes due to temperature, drift, and atmospheric pressure all have a time dependency, which means, in theory these changes may have some effect on when dP/dt=0 or when dP/dt peaks. However, the largest discrepancies caused by temperature and drift will occur when the catheter is first being introduced in the body, as this is when the sensor is transitioning from a dry state at room temperature to a “wet” state at body temperature. By the time the catheter is deployed/positioned and data starts to be analyzed, the amplitudes and frequencies of the changes due to temperature, drift, and atmospheric pressure are all be minimal compared to the amplitude and frequencies of the pressures in the heart. Therefore, even without correcting for changes due to temperature, drift, and atmospheric pressure, the effects to dP/dt=0 or when dP/dt peaks should be negligible.

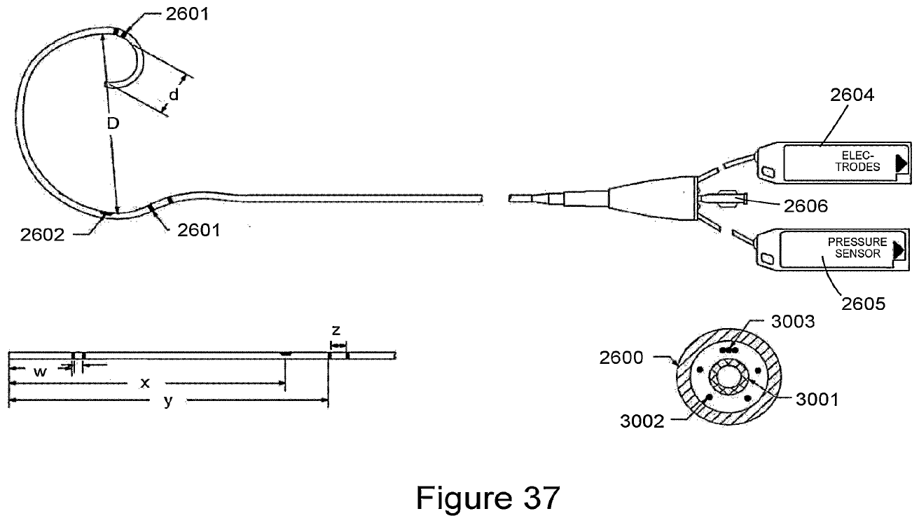

An exemplary catheter is shown in FIG. 37, along with some example dimensions over which it may extend. In order to provide electrodes 2601 and sensors 2602 at desired positions within the hear, the flexible tip may be provided at a small diameter, d. The middle part of the catheter may be provided at a larger diameter, D. As an example, diameter d may be in the order of 1.5 cm, and diameter D may be in the order of 6 cm. The total length of the catheter may be in the order of 130 cm. Electrodes 2601 closest to the tip of the catheter may be 1 mm wide, and may be positioned at a distance w from the tip, for example 3 cm. The two electrodes disposed closest to the tip may be disposed 8 mm apart. Sensor 2602 may be provided at a distance x from the tip of the catheter, for example 11 cm. Further electrodes 2601 may provided at a distance y from the tip of the catheter, for example 13 cm. Said electrodes may be provided at a distance z apart, again this may be for example 8 mm. Of course, said dimensions are exemplary, and other dimensions are envisioned.

In sum, in the above system, the distal segment of the catheter is adapted to be positioned with electrodes opposing each other in the heart. The distal segment has an area intended to contact the heart tissue. The distal segment carries one or more electrodes and one or more sensors (for example a pressure sensor, piezoelectric sensor, fiberoptic sensor, accelerometer) located proximal on the distal end of the catheter. The sensor(s) provide data on cardiac contraction, onset of synergy, valve events, pressure to a receiver connected to the processor. The electrodes connect to an amplifier that connect to a processor. The electrodes connect to a stimulator. The processor may analyse the data received to determine a point relating to the onset of synergy, and utilise this to determine if dyssynchrony and dyssynergy is present, and then further if stimulating the electrodes results in reversal of dyssynchrony and dyssynergy.

When the catheter is suitably positioned in the left heart chamber with electrodes opposing each other at the septum and contralateral wall and the sensor within the chamber, with each heartbeat a voltage gradient is registered between each electrode and a reference electrode. Such a voltage gradient represents electric activation of the heart. Further, and following on from the above, the sensor(s) register events related to the onset of synergy, i.e. events that relate to the rapid increase in rate of pressure rise within the left ventricle, which reflects the point where all segments of the heart begin to actively or passively stiffen to a maximal extent. The time to this event is compared with electrical activation, and the presence or absence of dyssynchrony and dyssynergy is registered.

The heart can then be stimulated from one or more electrode. With each heartbeat a voltage gradient is registered between each electrode and a reference electrode, which as described above can represent the electric activation of the heart. The one or more sensor again registers events related to the onset of synergy. The new set of time events may then be compared to the first set of events and the presence or absence of resynchronization is registered.

Advantageously, with such a system, it may be possible to quickly and efficiently determine such measures for various positions of electrodes. In this way, not only may it be determined if a patient is indeed a potential responder for cardiac resynchronisation therapy, but also the ideal number and positions of electrodes may be quickly determined.

1. A catheter for assessing cardiac function, the catheter comprising:
an elongate shaft extending from a proximal end to a distal end, the shaft comprising:
a lumen for a guidewire and/or a saline flush;
at least one electrode disposed on the shaft for sensing electrical signals in a bipolar or unipolar fashion and applying pacing to a patient's heart;
at least one sensor disposed on the shaft for detecting an event relating to the rapid increase in the rate of pressure increase within the left ventricle of a patient; and
communication means configured to transmit data received from the electrode(s) and the sensor(s).

2. The catheter of claim 1, wherein the at least one sensor comprises a pressure sensor, a piezoelectric sensor, a fiberoptic sensor, and/or an accelerometer.

3. The catheter of claim 1, wherein the stiffness of the elongate shaft varies along its length between the proximal end and the distal end.

4. The catheter of claim 3, wherein the elongate shaft is provided with a stiff proximal end, a middle part which is of an intermediate stiffness, and a flexible tip at the distal end.

5. The catheter of claim 1, wherein the at least one electrode comprises a plurality of electrodes disposed along the shaft such that, in use, at least two electrodes may be positioned opposing each other in the heart of the patient.

6. The catheter of claim 5, wherein at least one electrode is configured to be placed within the septum of the patient, and at least one electrode is configured to be placed in the contralateral wall of the patient.

7. A system comprising:
the catheter of claim 1;
a signal amplifier;
a stimulator; and
a data processing module;
wherein the catheter is configured to be in signal communication with the stimulator, the amplifier and data processing module such that the electrode(s) and sensor(s) may provide sensed data to the data processing module for further processing, and the electrode(s) may provide pacing to the patient's heart.

8. The system of claim 7, wherein the data processing module is configured to determine a characteristic response relating to the onset of myocardial synergy from the event relating to the rapid increase in the rate of pressure increase within the left ventricle of a patient.

9. The system of claim 8, wherein the sensor(s) are configured to provide data regarding the pressure within the heart to the data processing module, and wherein the data processing module is configured to filter the pressure data to identify the characteristic response relating to the onset of myocardial synergy.

10. The system of claim 9, wherein the characteristic response comprises (i) the beginning of a pressure rise above the pressure floor in a pressure signal filtered above the first harmonic of the pressure signal or (ii) the presence of high frequency components (above 40 Hz) of the pressure signal or (iii) a band-pass filtered pressure trace crossing zero.

11. (canceled)

12. (canceled)

13. The system of claim 8, wherein the sensor(s) are configured to provide acceleration data from within the heart to the data processing module, and wherein the data processing module is configured to filter the acceleration data to identify a characteristic response relating to the onset of myocardial synergy.

14. The system of claim 13, wherein the data processing module is configured to calculate (i) a continuous wavelet transform of the acceleration data to identify a characteristic response relating to the onset of myocardial synergy or (ii) the center frequency of the continuous wavelet transform, wherein the characteristic response is the peak of the center frequency, and wherein the data processing module is configured to average the center frequency over a number of heart cycles.

15. (canceled)

16. (canceled)

17. The system of claim 8, wherein the data processing module is configured to identify reversible cardiac dyssynchrony by identifying a shortening of a delay to onset of myocardial synergy as a result of pacing.

18. The system of claim 17, wherein the data processing module is configured to identify reversible cardiac dyssynchrony of a patient using the at least one sensor to measure the time of the event relating to the rapid increase in the rate of pressure increase within the left ventricle of a patient by identifying the characteristic response in the data received from the one or more sensors, the event relating to the rapid increase in the rate of pressure increase within the left ventricle being identifiable in each contraction of the heart, the data processing module being configured to measure the time of the event relating to the rapid increase in the rate of pressure increase within the left ventricle by;
processing signals from the at least one sensor to determine a first time delay between the measured time of the identified characteristic response relating to the rapid increase in the rate of pressure increase within the left ventricle and a first reference time;
comparing the first time delay between the measured time of the identified characteristic response relating to the rapid increase in the rate of pressure increase within the left ventricle and the first reference time with the duration of electrical activation of the heart;
if the first time delay is longer than a set fraction of electrical activation of the heart, then identifying the presence of cardiac dyssynchrony in the patient;
following the application of pacing by the at least one electrode and/or other electrodes to the heart of the patient;
calculate a second time delay between the identified characteristic response relating to the rapid increase in the rate of pressure increase within the left ventricle following pacing and a second reference time following pacing by:
using the at least one sensor to measure the timing of the identified characteristic response relating to the rapid increase in the rate of pressure increase within the left ventricle following pacing; and
processing signals from the at least one sensor to determine the second time delay between the determined time of the identified characteristic response relating to rapid increase in the rate of pressure increase within the left ventricle and the second reference time following pacing;
compare the first time delay and the second time delay; and
if the second time delay is shorter than the first time delay, identifying a shortening of a delay to onset of myocardial synergy, OoS, indicating that the time period until the point where all segments of the heart begin to actively or passively stiffen has shortened, thereby identifying the presence of reversible cardiac dyssynchrony in the patient.

19. The system of claim 18, wherein the data processing module is further configured to, if the first time delay is shorter than a set fraction of electrical activation of the heart, then identify the absence of cardiac dyssynchrony in the patient; and/or
if the first time delay is shorter than a set delay, for example 120 ms, then identify the absence of cardiac dyssynchrony in the patient.

20. The system of claim 7, wherein the data processing module is configured to determine the degree of parallel activation of a heart undergoing pacing.

21. The system of claim 20, wherein the data processing module is configured to determine the degree of parallel activation of a heart undergoing pacing via a method comprising:
calculating a vectorcadiogram, VCG, or electrocardiogram, ECG, waveforms from right ventricular pacing, RVp, and left ventricular pacing, LVp;
generating a synthetic biventricular pacing, BIVP, waveform pacing by summing the VCG of the RVp and the LVp, or by summing the ECG of the RVp and the LVp;
calculating a corresponding ECG or VCG waveform from real BIVP;
comparing the synthetic BIVP waveform and the real BIVP waveform;
calculating time to fusion by determining the point in time in which the activation from RVp and LVp meets and the synthetic and the real BIVP curves start to deviate;
wherein
a delay in time to fusion indicates that a larger amount of tissue is activated before wave fronts for electrical activation meet, thereby indicating a higher degree of parallel activation.

22. The system of claim 7, wherein the data processing module is configured to determine the optimal electrode number and position for cardiac resynchronization therapy on the heart of the patient based on node(s) of a 3D mesh 3D mesh of at least part of the heart with a calculated degree of parallel activation of the myocardium above a predetermined threshold.

23. The system of claim 22, wherein the determining optimal electrode number and positions for cardiac resynchronization therapy on a heart of a patient, is performed via a method comprising;
generating the 3D mesh of at least part of the heart from a 3D model of at least part of the heart of the patient, or using a generic 3D model of the heart to obtain a 3D mesh of at least a part of the heart, the 3D mesh of at least a part of the heart comprising a plurality of nodes;
aligning the 3D mesh of at least part of a heart to images of the heart of the patient;
placing additional nodes onto the 3d mesh corresponding to a location of at least two electrodes on the patient;
calculating a propagation velocity of the electrical activation between the nodes of the 3D mesh corresponding to the location of the at least two electrodes;
extrapolating the propagation velocity to all of the nodes of the 3D mesh;
calculating the degree of parallel activation of the myocardium for each node of the 3D mesh; and
determining the optimal electrode number and position on the heart of the patient based on the node(s) of the 3D mesh with a calculated degree of parallel activation of the myocardium above a predetermined threshold.

24. The system of claim 7, wherein the catheter is configured to be provided into a patient's heart through arterial access, venal access, subclavian access, radial access and/or femoral access such that the electrode(s) and sensor(s), in use, may be provided within the heart of the patient.
